(19) United States
(12) Patent Application Publication
(10) Pub. No.: US 2023/0042456 A1
(43) Pub. Date: Feb. 9, 2023

(54) INCREASING SPACE-TIME-YIELD, CARBON-CONVERSION-EFFICIENCY AND CARBON SUBSTRATE FLEXIBILITY IN THE PRODUCTION OF FINE CHEMICALS

(71) Applicant: BASF SE, Ludwigshafen am Rhein (DE)

(72) Inventors: Hartwig SCHROEDER, Ludwigshafen am Rhein (DE); Anne-Catrin HAILER, Ludwigshafen am Rhein (DE); Birgit HOFF, Ludwigshafen am Rhein (DE); Oskar ZELDER, Ludwigshafen am Rhein (DE); Peter OEDMAN, Ludwigshafen am Rhein (DE); Georg Benjamin WANDREY, Ludwigshafen am Rhein (DE); Christian DIETZSCH, Ludwigshafen am Rhein (DE); Mukesh KUMAR, Tarrytown, NY (US); Dominik SATORY, Tarrytown, NY (US); Matthew David BLANKSCHIEN, Tarrytown, NY (US); Jens Klaus PLASSMEIER, Westford, MA (US)

(21) Appl. No.: 17/786,864

(22) PCT Filed: Dec. 16, 2020
(86) PCT No.: PCT/EP2020/086342

§ 371 Date: Jun. 17, 2022

Related U.S. Application Data
(60) Provisional application No. 62/950,167, filed on Dec. 19, 2019.

(30) Foreign Application Priority Data
Dec. 19, 2019 (EP) 19217809.3
Aug. 28, 2020 (EP) 20193397.5

(51) Int. Cl. C12P 19/04; C12N 9/88

(52) U.S. Cl. CPC C12P 19/04; C12N 9/88; C12Y 406/01001

(57) ABSTRACT

Increasing space-time-yield, carbon-conversion-efficiency and carbon substrate flexibility in the production of fine chemicals The inventors of the current invention have found a surprising positive effect of increased cAMP levels and/or manipulating the PTS system on the space-time-yield, carbon-conversion-efficiency and carbon substrate flexibility of fine chemical production of a host organism. This was achieved by de-regulating adenylate cyclase cyaa by deleting the C-terminal regulatory region leading to increased cAMP levels or deleting the Crr protein activity (carbohydrate repression resistance) which regulates the carbohydrate utilization system. Both lead to increased 2-fucosyllactoe and 6-sialyllactose production (human milk oligosaccharides) and increase carbohydrate usage.

The inventors of the current invention have found a surprising positive effect of increased cAMP levels on the space-time-yield, carbon-conversion-efficiency and carbon substrate flexibility of fine chemical production of a host organism. Moreover, the inventors have found that an adenylate cyclase activity that is not subject to its endogenous regulation, and hence is always active in cAMP production is beneficial for the space-time-yield and carbon substrate flexibility of fine chemical production by a host organism.

Furthermore, the inventors of the current invention have also found a surprising effect of a decreased expression of the crr gene or variant thereof and/or an inactivation of or reduction of the Crr protein or variants thereof on the carbon conversion efficiency, carbon substrate flexibility and space/time of the production of oligosaccharides by a prokaryotic organism.

The Crr protein is part of the PTS carbohydrate utilization system of microbes, which is also linked to the cAMP levels in the microbial cell.

It is known from the state of the art that decreasing the expression of proteins of the PTS carbohydrate utilization system (PTS system) has an effect on the production of certain compounds other than oligosaccharides.

Flores et al. (Nature Biotechnology (1996), Volume 14, pages 620-623) describes the pathway engineering for the production of aromatic compounds in Escherichia coli. A theoretical analysis of the pathways involved in the production of aromatic compounds in E. coli indicates that the yield of this compounds is limited by phosphoenolpyruvate (PEP) availability. This compound is one of the major building blocks in several biosynthetic pathways, and it is the donor utilized in the PTS system in the internalization of glucose. Two molecules of PEP are produced from one mol glucose from the glycolytic pathway. One mol if PEP, however, subsequently used by the PTS system during glucose transport, leaving only one mol of PEP per mol of glucose consumed that is available for other metabolic reactions. Flores at all. Found that when E. coli strains devoid of the ptsH, ptsl and crr genes are cultivated in a fermentor in a minimal medium with glucose as the only carbon source, a heterogeneous population of PTS-Glucose+revertants can be detected after two days. These revertant are able to transport Glucose trough GaIP, and one in the cytoplasm, the glycose is phoshorylated by glucokinase using ATP. A further aspect of the invention relates to the combination of an adenylate cyclase activity that is not subject to its endogenous regulation and a decreased expression of the crr gene or variant thereof and/or an inactivation of or reduction of the Crr protein or variants thereof and the effect of this combination within one host cell on the carbon conversion efficiency, carbon substrate flexibility and space/time of the production of oligosaccharides by a prokaryotic host organism.

### DETAILED DESCRIPTION OF THE INVENTION

Space-time-yield is defined as the rate of product formation per time. It can be related to the space or amount of the reaction mixture or fermentation defined by either its volume or its weight. Typical definitions include weight e.g. gram of product produced per volume (like litre) or weight (like kg) of fermentation broth per time unit (like hour).

Increasing space-time-yield of a given fine chemical as product is increasing the productivity of the specific product by increasing the rate of product formation defined by its volume or weight over time in a given reaction space. During a given period, a larger amount of the fine chemical product is being produced with the same set-up when the space-time-yield is increased. The same amount of fine chemical can also be produced in a given set-up in a shorter time when the space-time-yield is increased.

Carbon-conversion-efficiency is known as the ratio of specific product formation as an amount per amount of carbon source consumed. It can be related to molar ratios e.g. moles of product produced per moles of carbon source consumed. Also, carbon-conversion-efficiency can be described as the ratio of functional moiety in the final molecule per molecule of product.

In a preferred definition the carbon-conversion-efficiency according to the invention is defined as the weight of the specific product produced per weight of carbon source being used in the process This calculation can be advantageous since carbon-conversion-efficiency using different carbon sources having different molecular weights (e.g. maltose, glucose, mannose, glycerol, sucrose, gluconate) can be compared directly.

Moreover, the carbon-conversion-efficiency of the production of fine chemicals is increased by the methods of the invention and in the host cells of the invention. With the increased cAMP host cells, an increased percentage of carbon atoms fed to the cells is channelled into the desired fine chemical product, and hence less carbon is lost due to unwanted side reactions or to carbon dioxide via cellular respiration. On the road to a more climate friendly economy, a reduced loss of carbon to carbon dioxide is desirable.

Preferably the carbon-conversion-efficiency and/or space-time-yield is increased by 1, 2, 3 . . . percent, more preferably by 4, 5, 6, 7, 8, 9 or 10% compared to the control, i.e. the unmodified cell holding only normally regulated adenylate cyclase.

More preferably, the carbon-conversion-efficiency and/or space/time yield is improved by a factor of 1.1, 1.2, 1.25, 1.3, 1.4, 1.5, 1.75, 2, 3, 4, 5, 6, 7, 8, 9, or 10.

Methods to increase the carbon-conversion-efficiency of the production of one or more fine chemicals by a host organism are also part of the invention, wherein the cAMP levels in the host organism is increased compared to the non-modified host organisms.

Carbon substrate flexibility is defined by the ability of a host cell to use more than one specific carbon source. Typical carbon sources suitable for a fine chemical producing strain can be found in Escherichia coli (E. coli) and Salmonella: Cellular and Molecular Biology ASM press 1996. As used throughout this text, increased carbon substrate flexibility is the characteristic of a modified host cell to grow on a carbon source that the unmodified host cell is unable to grow on or to grow substantially better on a carbon source than the control, which maybe a wildtype cell or the unmodified host cell.

Carbon sources are batched into the medium and/or fed during the feed phase. Typical fine chemical production periods are ranging from 24 h- to 100 h.

The cAMP level of the host organism is preferably the intracellular cAMP level, and more preferably the cytoplasmic cAMP level of a host organism.

cAMP level s can be determined by a number of methods known in the art, for example using cAMP specific antibodies that then can be used with a range of detection methods including luciferase-based assays. Commercial kits for measuring cAMP levels in cells, tissues and biological samples are available (for example from Sigma Aldrich CA200 cAMP Enzyme Immunoassay Kit). Other methods for the determination of cAMP can be found in: Crasnier 1990, Journal of General Microbiology 136: 1825-1831, in: Guidi-Rontani et al. 1981 J. Bacteriology 148:753-761, or in: J. Chromatogr. B. Analyt. Technol. Biomed. Life Sci. 2012 909:14-21.

In one embodiment, the cAMP levels are increased by external addition of cAMP and/or by import or re-import of cAMP into the host cell. In another embodiment, cAMP level of the host organism is increased by the steps of inactivating the regulatory activity found in a wildtype adenylate cyclase, and/or introducing a mutated adenylate cyclase lacking the regulatory activity found in a wildtype adenylate cyclase. In another embodiment the level of cAMP can be increased by reduction of the activity of the enzyme with the activity of a 3′,5′ cAMP phosphodiesterase (EC 3.1.4.53) and optionally other diesterases like those of enzyme class EC 3.1.4.17 or EC 3.1.4.16 when acting on 3,5 cAMP. Activity reduction can be achieved for example by knock-out of the gene, Antisense or RNAi techniques, introduction of activity reducing or activity abolishing mutations or by inhibitors. An example of a 3′,5′ cAMP phosphodiesterase is the one encoded by the gene cpdA of Escherichia coli. Another way to increase the cAMP levels in the cell is by the use of adenylate cyclase domain of the adenylate cyclase toxin of Bordetella pertussis or the full adenylate cyclase toxin protein.

The methods of the invention are methods for the increase of space-time-yield of one or more fine chemicals produced by a host organism as well as for the increase of carbon substrate flexibility and the carbon-conversion-efficiency of the production of one or more fine chemicals by a host organism compared to the non-modified host organisms including the steps of providing a host organisms capable of producing the one or more fine chemicals, increasing the Adenosine 3′,5′-cyclic monophosphate (cAMP, CAS Number: 60-92-4) levels of the host organism, maintaining the host organism in a setting allowing it to grow, growing the host organisms in the presence of substrates and nutrients and under conditions suitable for the production of one or more fine chemicals and optionally separating one or more fine chemicals from the host organism or remainder thereof, wherein the host organism is suitable to produce said one or more fine chemicals in the non-modified and the modified form.

The cAMP level of the host organism in one embodiment are increased in an inducible manner and the increase is compared to the host organisms without such induction. Methods for the inducer dependent gene expression for example by the inducer Isopropyl β-d-1-thiogalactopyranoside (IPTG) are known in the art.

In a preferred embodiment, the increased cAMP levels can be achieved by providing in the host cell an adenylate cyclase protein with inactive, inhibited or missing regulatory domain (referred to herein as inactive regulatory domain or inactive regulatory part) and functional catalytic domain to produce cAMP. The inactive regulatory domain can be inactive due to the presence of an inhibitor, or due to an inactivating mutation or due to deletion in whole or part of the regulatory domain of the adenylate cyclase protein. The absence of part or all of the regulatory domain of the adenylate cyclase protein can be achieved by any number of means, for example by introducing a copy of the adenylate cyclase gene that is truncated, as shown in numerous ways in this invention, or by altering the mRNA of adenylate cyclase or by premature termination of protein translation of the transcript or by removal of part or all of the regulatory domain after translation.

The enzyme adenylate cyclase is also called 3′,5′-cyclic AMP synthetase, Adenyl cyclase, Adenylyl cyclase or ATP pyrophosphate-lyase.

The international patent application published as WO 98/29538 disclosed an adenylate cyclase gene of Ashbya gossypii and that said adenylate cyclase gene may be used in microorganisms for the production of fine chemicals such as riboflavin. Further it was disclosed in said application that the production of riboflavin by the fungus Ashbya gossypii grown on glucose containing media is increased when the endogenous adenylate cyclase gene has been disrupted in the Adenosine 3′,5′-cyclic monophosphate (3′,5′-cyclic AMP or cAMP, CAS Number: 60-92-4) producing part. Also disclosed is that increasing cAMP levels by addition of cAMP has a negative effect on riboflavin production in the disrupted strain.

It has been shown that altering the activity of the adenylate cyclase has an effect on the uptake of carbon sources either utilizing the so called phospotransferase system (PTS) or using other mechanisms are influenced by mutations in the cyaA gene coding for the adenylate cyclase. It has been shown that mutations in cyaA confer an inability to utilize carbon sources such as lactose, maltose, arabinose, mannitol or glycerol, and ferments weakly and grows slowly on glucose, fructose and galactose (Perlman R, et al. 1969 Biochemical and Biophysical Research Communications 37(1), pp. 151-157),

It has not been shown previously that the production of fine chemicals, specifically oligosaccharides is positively influenced by an alteration of the cyaA gene that increases the synthesis of cAMP.

As described above, inactivating the regulatory activity found in a wildtype adenylate cyclase can be achieved in a number of ways, for example by the use of an inhibitor, or due to an inactivating mutation or due to deletion in whole or part of the regulatory domain of the adenylate cyclase wildtype protein, for example by altering or deleting in part the mRNA coding for adenylate cyclase in the host organisms, the mRNA translation of the adenylate cyclase or by mutating or deleting a gene sequence encoding the regulatory part of the adenylate cyclase. For example, CRISPR/CAS technology (Wang, H H. (2013), Mol. Syst. Biol. 9 (1): 641) may be used to specifically eliminate or replace in a non-functional manner the part of the gene sequence of the adenylate cyclase that is responsible for the regulatory part of the adenylate cyclase protein. The international patent application published as WO2011102305 discloses a specific mutation to Leucine at position 432 of the cyaA gene of E. coli to be useful in the production of amino acids. Reddy et al. (Analytical Biochemistry 231, 282-286 (1995)) and Crasnier et al. (J. Gen. Microbiol. 1990; 136:1825-31, Mol. Gen. Genet. 1994; 243:409-16) disclose that the catalytic domain of E. coli adenylate cyclase is in the N-terminal part of the protein and that deletions in the C-terminal part may increase the adenylate cyclase activity or may interfere with the negative regulation by effectors. Lindner (Biochem. J. (2008), 415, 449-454) discloses results on the detailed study of the residues in the catalytic part of E. coli adenylate cyclase comprising amino acid positions 1 to 412.

Preferably the regulatory part or domain is defined as that part of the protein harbouring adenylate cyclase activity that is not directly involved in the production of cAMP but controls the activity of the cAMP producing part that contains the active site.

An adenylate cyclase producing part useful in the methods and host cells of the invention is a protein or part thereof with an enzymatic activity of EC 4.6.1.1 and has the ability to produce Adenosine 3′,5′-cyclic monophosphate (cAMP).

In E. coli cells two variants of the adenylate cyclase protein and genes encoding such were found. One is the widely found protein with a length of 848 amino acids (SEQ ID NO: 19, encoded by the nucleotide sequence provided as SEQ ID NO: 9), and a variant of this full-length protein that has a duplication of 6 amino acids and hence has 854 amino acids (SEQ ID NO: 20, encoded by the nucleotide sequence provided as SEQ ID NO: 10). In the longer variant, the amino acid motif GEQSMI is present as a duplicate (see FIG. 2, part 2 underlined stretch of amino acids), while the variant with 848 amino acids contains this motif only one time. This motif is part of the PFAM domain PF01295 that is found in adenylate cyclases. In the present invention it is disclosed that de-regulated version of either of these two variants of adenylate cyclase of E. coli results in increased space/time yield, carbon-conversion-efficiency and carbon source flexibility.

Within the context of this invention the cyaA gene of Escherichia coli is understood to be any of the genes shown in SEQ ID NO 9 or 10 or a DNA encoding the protein sequence of SEQ ID NO: 19 or 20 or a protein with 70% identity, preferably at least 75%, at least 80%, at least 85%, at least 90%, more preferably at least 95%, at least 97%, at least 98% or at least 99% over the full length of either one of SEQ ID NOs: 19 or 20, and most preferably encoding a protein with adenylate cyclase activity, i.e. activity of EC 4.6.1.1.

Truncated adenylate cyclase proteins with reduced or inactivate regulatory part but cAMP forming activity are beneficial in the methods and host cells of the invention.

Particularly useful in the methods and host cells of the invention are adenylate cyclase proteins corresponding to the protein encoded by the cyaA gene of Escherichia coli yet lacking the regulatory activity, preferably lacking the part that corresponds to C-terminal part of the CyaA protein as provided in SEQ ID NOS:19 or 20, or an adenylate cyclase protein of at least 60%, 65%, 70%, 75%, 80%, 85%, 90%, 95% or 98% sequence identity to positions 1 to 412 of the protein sequence provided as SEQ ID NO 19 or 20, more preferably to positions 1 to 420, of the protein sequence provided as SEQ ID NO 19 or 208, and preferably lacking the part of the Escherichia coli adenylate cyclase that is subsequent to position 420, 450, 558, 585, 653, 709, 736 or 776, more preferably 450, 558, 585, 653, 709 or, 736 of the protein sequence supplied in SEQ ID Nos: 19 or 20 even more preferably subsequent to position 558, 582, 585, 653, 709, 736 or 776 of the protein sequence supplied in SEQ ID Nos: 19 or 20. Subsequent to a given position is to be understood as all of the amino acids found in the protein of interest following the amino acid that corresponds to the given position in SEQ ID NO: 19 or 20.

A table of exemplary shortened adenylate cyclase proteins and genes are shown in table 1.

TABLE 1
Overview of full-length and shortened adenylate cyclase proteins and
genes of the sequence listing. FL is the abbreviation for full-length

| | | | Protein contains |
|---|---|---|---|
| | | | regulatory part |
| Protein | DNA SEQ ID NO: | Protein SEQ ID NO: | of protein |
| cyaA420 | 1 | 11 | No |
| cyaA450 | 2 | 12 | No |
| cyaA558 | 3 | 13 | No |
| cyaA585 | 4 | 14 | No |
| cyaA653 | 5 | 15 | No |
| cyaA709 | 6 | 16 | No |
| cyaA736 | 7 | 17 | No |
| cyaA776 | 8 | 18 | No |
| FL cyaA | 9 & 10 | 19 & 20 | YES |

The shortened proteins cyaA653, cyaA709, cyaA736 and cyaA776 (SEQ ID NOs: 15 to 18) contain the duplicate GEQSMI motif as found in the full-length version of 854 amino acids (SEQ ID NO: 20). The other shortened versions do not carry the motif at all. The advantageous effects in the methods and host cells of the present invention were found to be independent of the presence of the single or the duplicate GEQSMI motif as shown in the examples section below in detail.

In a preferred embodiment the methods of the invention are methods for the increase of spacetime-yield of one or more fine chemicals produced by a host organism as well as for the increase of carbon substrate flexibility and the carbon-conversion-efficiency of the production of one or more fine chemicals by a host organism including the steps of providing a host organisms capable of producing the one or more fine chemicals, providing a de-regulated adenylate cyclase capable of producing cAMP in the host organism, maintaining the host organism in a setting allowing it to grow, growing the host organisms in the presence of substrates and nutrients and under conditions suitable for the production of one or more fine chemicals and optionally separating one or more fine chemicals from the host organism or remainder thereof.

In an embodiment the de-regulated adenylate cyclase protein useful in the methods and host cells of the inventions, is an enzyme of adenylate cyclase activity without the regulatory part found in the wildtype adenylate cyclase protein of the host cell. Preferably it is the adenylate cyclase protein of the host cell—or variants or part thereof that are active adenylate cyclase enzymes but not subject to at least some of the regulatory mechanisms as the unmodified adenylate cyclase of said host cell is—and corresponding to the E. coli adenylate cyclase as provided in SEQ ID NOs: 19 or 20. Preferably the de-regulated adenylate cyclase useful in the methods and host cells of the invention is lacking the part that corresponds to the C-terminal part of the CyaA protein as provided in SEQ ID NOS:19 or 20, or is an adenylate cyclase protein of at least 80% sequence identity to positions 1 to 412, more preferably an adenylate cyclase protein of at least 80% sequence identity to positions 1 to 420, of the protein sequence provided as SEQ ID NO 19 or 20. More preferably it is lacking the part of the adenylate cyclase that corresponds to the Escherichia coli adenylate cyclase part that is subsequent to position 420, 450, 558, 585, 653, 709, 736 or 776, more preferably subsequent to positions 450, 558, 585, 653, 709 or 736 of the protein sequence supplied in SEQ ID Nos: 19 or 20, even more preferably subsequent to position 558, 582, 585, 653, 709, 736 or 776 of the protein sequence supplied in SEQ ID Nos: 19 or 20, and most preferably lacking the amino acids that correspond to the amino acids at the position 777 and following of SEQ ID NO 19 or 20. In another preferred embodiment the de-regulated adenylate cyclase protein, is the part of the endogenous adenylate cyclase of a host organisms that corresponds to any of the sequences of SEQ ID NO: 11 to 18 and more preferably is any of the sequences provided as SEQ ID NO: 11 to 18, or is encoded by any of the sequences of SEQ ID NO:1 to 8, or variants thereof, including proteins with tags and fusion proteins comprising the de-regulated adenylate cyclase. In one embodiment also included are amino acid sequences with one to several amino acid changes compared to the sequences of SEQ ID NO: 11 to 18, as long as these have adenylate cyclase activity without a regulation of said activity as found in the unmodified CyaA protein of the host cell corresponding to the proteins of SEQ ID NO 19 or 20. Preferably the de-regulated adenylate cyclase results in increased cAMP levels of the host cell that is increased.

Preferably, such variants of amino acids sequences do not comprise a substitution of the L-lysine residue in the adenylate cyclase part by a L-glutamine at the position corresponding to position 432 of the sequence disclosed as SEQ ID NO: 2 in the international application published as WO2011102305.

The modified host cell holding a de-regulated adenylate cyclase protein can be achieved by a number of means, such as mutation and selection, recombinant methods for example introduction of a shortened cyaA gene and gene editing methods like CRISPR/CAS.

The host cell of the invention or useful in the methods of the invention is preferably a bacterial or fungal host cell, more preferably a bacterial cell selected among the group consisting of gram-positive and gram-negative bacteria or a yeast cell, even more preferably it is selected from the genera Bacillus, Clostridium, Enterobacteriaceae, Enterococcus, Erwinia, Escherichia, Klebsiella, Lactobacillus, Lactococcus, Mycoplasma, Pasteurella, Rhodobacter, Rhodoseudomonas, Salmonella, Staphylococcus, Streptococcus, Vibrio, and Xanthomonas, or a yeast cell of the genus Pichia, Kluveromyces or Saccharomyces, yet even more preferably an E. coli cell, a Corynebacterium sp. cell or a Saccharomyces sp. cell.

In one embodiment the host cell of the invention is a bacterial or fungal host cell, preferably a bacterial cell, preferably a cell utilizing cAMP for regulation of cellular pathways, more preferably a cell harbouring a functional adenylate cyclase more preferably proteobacterium, a gamma proteobacterium, a bacterium of the family of Enterobacteriaceae, even more preferably bacterium of the genus Escherichia and yet even more preferably a bacterium of the species Escherichia coli.

Fine chemical according to the invention is a biochemical substance comprising two or more sugar units. Preferably, the fine chemical is a biochemical substance produced by a genetically modified organism. More preferably, the fine chemical of the invention comprises or consists of one or more oligosaccharides. Even more preferably, the fine chemical produced by the host cells and methods of the invention comprises or consists of a human milk oligosaccharide (HMO), even more preferably a neutral or sialylated HMO, even more preferably fucosylated or sialylated HMO, and yet even more preferably the fine chemical is 3′-sialyllactose (3′-SL), 6′-sialyllactose (6′-SL), 2′-fucosyllactose (2′-FL), difucosyllactose (2,3-DFL), 3′-fucosyllactose (3′-FL), Lacto-N-triose, Lacto-N-Tetraose (LNT) or lacto-N-neotetraose (LNnT). Examples for human milk oligosaccharides can be found in Niñonuevo M R et al. (2006). J. Agric. Food Chem. 54:7471-7480, Bode L (2009) Nutr. Rev. 67:183-191, Bode L (2012) Glycobiology 22:1147-1162, Bode L (2015) Early Hum. Dev. 91:619-622.

In a most preferred embodiment the fine chemical of the invention is 2′-FL or 6′-SL.

### Terms and Meaning

Unless otherwise noted, the terms used herein are to be understood according to conventional usage by those of ordinary skill in the relevant art. In addition to the definitions of terms provided herein, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement).

It is to be understood that as used in the specification and in the claims, “a” or “an” can mean one or more, depending upon the context in which it is used. Thus, for example, reference to “a cell” can mean that at least one cell can be utilized. It is to be understood that the terminology used herein is for the purpose of describing specific embodiments only and is not intended to be limiting.

In the description of the present invention, genes and proteins are identified using the denominations of the corresponding genes in Escherichia coli. However, and unless specified otherwise, use of these denominations has a more general meaning according to the inventions and covers all the corresponding genes and proteins in other organisms, particularly microorganisms.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in M. Green & J. Sambrook (2012) Molecular Cloning: a laboratory manual, 4th Edition Cold Spring Harbor Laboratory Press, CSH, New York; Ausubel et al., Current Protocols in Molecular Biology, Wiley Online Library; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York.

If not stated otherwise herein, abbreviations and nomenclature, where employed, are deemed standard in the field and commonly used in professional journals such as those cited herein.

The terms “essentially”, “about”, “approximately”, “substantially” and the like in connection with an attribute or a value, particularly also define exactly the attribute or exactly the value, respectively. The term “substantially” in the context of the same functional activity or substantially the same function means a difference in function preferably within a range of 20%, more preferably within a range of 10%, most preferably within a range of 5% or less compared to the reference function. In context of formulations or compositions, the term “substantially” (e.g., “composition substantially consisting of compound X”) may be used herein as containing substantially the referenced compound having a given effect within the formulation or composition, and no further compound with such effect or at most amounts of such compounds which do not exhibit a measurable or relevant effect. The term “about” in the context of a given numeric value or range relates in particular to a value or range that is within 20%, within 10%, or within 5% of the value or range given. As used herein, the term “comprising” also encompasses the term “consisting of”.

The term “isolated” means that the material is substantially free from at least one other component with which it is naturally associated within its original environment. For example, a naturally-occurring polynucleotide, polypeptide, or enzyme present in a living animal is not isolated, but the same polynucleotide, polypeptide, or enzyme, separated from some or all of the coexisting materials in the natural system, is isolated. As further example, an isolated nucleic acid, e.g., a DNA or RNA molecule, is one that is not immediately contiguous with the 5′ and 3′ flanking sequences with which it normally is immediately contiguous when present in the naturally occurring genome of the organism from which it is derived. Such polynucleotides could be part of a vector, incorporated into a genome of a cell with an unrelated genetic background (or into the genome of a cell with an essentially similar genetic background, but at a site different from that at which it naturally occurs), or produced by PCR amplification or restriction enzyme digestion, or an RNA molecule produced by in vitro transcription, and/or such polynucleotides, polypeptides, or enzymes could be part of a composition, and still be isolated in that such vector or composition is not part of its natural environment.

“Purified” means that the material is in a relatively pure state, e.g., at least about 90% pure, at least about 95% pure, or at least about 98% or 99% pure. Preferably “purified” means that the material is in a 100% pure state.

A “synthetic” or “artificial” compound is produced by in vitro chemical or enzymatic synthesis. It includes, but is not limited to, variant nucleic acids made with optimal codon usage for host organisms, such as a yeast cell host or other expression hosts of choice or variant protein sequences with amino acid modifications, such as e.g. substitutions, compared to the wildtype protein sequence, e.g. to optimize properties of the polypeptide.

The term “non-naturally occurring” refers to a (poly)nucleotide, amino acid, (poly)peptide, enzyme, protein, cell, organism, or other material that is not present in its original environment or source, although it may be initially derived from its original environment or source and then reproduced by other means. Such non-naturally occurring (poly)nucleotide, amino acid, (poly)peptide, enzyme, protein, cell, organism, or other material may be structurally and/or functionally similar to or the same as its natural counterpart.

The term “native” (or “wildtype” or “endogenous”) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention). In one aspect, a wildtype adenylate cyclase is to be understood as a protein with adenylate cyclase activity (EC 46.1.1 comprising its normal regulatory part or domain and subject to the regulation as found in nature.

“Homologous” refers to a gene, polypeptide, polynucleotide with a high degree of similarity, e.g. in position, structure, function or characteristic, but not necessarily with a high degree of sequence identity. “Homologous” is not to be used interchangeably with “endogenous” or as an antonym of “heterologous” (see below).

The term “heterologous” (or exogenous or foreign or recombinant) polypeptide is defined herein as:
(a) a polypeptide that is not native to the host cell. The protein sequence of such a heterologous polypeptide is a synthetic, non-naturally occurring, “man-made” protein sequence;
(b) a polypeptide native to the host cell in which structural modifications, e.g., deletions, substitutions, and/or insertions, have been made to alter the native polypeptide; or
(c) a polypeptide native to the host cell whose expression is quantitatively altered or whose expression is directed from a genomic location different from the native host cell as a result of manipulation of the DNA of the host cell by recombinant DNA techniques, e.g., a stronger promoter.

Descriptions b) and c), above, refer to a sequence in its natural form but not naturally expressed by the cell used for its production. The produced polypeptide is therefore more precisely defined as a “recombinantly expressed endogenous polypeptide”, which is not in contradiction to the above definition but reflects the specific situation that it's not the sequence of a protein being synthetic or manipulated but the way the polypeptide molecule is produced.

Similarly, the term “heterologous” (or exogenous or foreign or recombinant) polynucleotide refers:
(a) to a polynucleotide that is not native to the host cell;
(b) a polynucleotide native to the host cell in which structural modifications, e.g., deletions, substitutions, and/or insertions, have been made to alter the native polynucleotide;
(c) a polynucleotide native to the host cell whose expression is quantitatively altered as a result of manipulation of the regulatory elements of the polynucleotide by recombinant DNA techniques, e.g., a stronger promoter; or
(d) a polynucleotide native to the host cell but integrated not within its natural genetic environment as a result of genetic manipulation by recombinant DNA techniques.

With respect to two or more polynucleotide sequences or two or more amino acid sequences, the term “heterologous” is used to characterize that the two or more polynucleotide sequences or two or more amino acid sequences do not occur naturally in the specific combination with each other.

The term “gene” means the segment of DNA involved in producing a polypeptide chain; it includes regions preceding and following the coding region (leader and trailer) as well as intervening sequences (introns) between individual coding segments (exons).

The term “gene” means a segment of DNA containing hereditary information that is passed on from parent to offspring and that contributes to the phenotype of an organism. The influence of a gene on the form and function of an organism is mediated through the transcription into RNA (tRNA, rRNA, mRNA, non-coding RNA) and in the case of mRNA through translation into peptides and proteins.

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

For nucleotide sequences, e.g., consensus sequences, an IUPAC nucleotide nomenclature (Nomenclature Committee of the International Union of Biochemistry (NC-IUB) (1984). “Nomenclature for Incompletely Specified Bases in Nucleic Acid Sequences”.) is used, with the following nucleotide and nucleotide ambiguity definitions, relevant to this invention: A, adenine; C, cytosine; G, guanine; T, thymine; K, guanine or thymine; R, adenine or guanine; W, adenine or thymine; M, adenine or cytosine; Y, cytosine or thymine; D, not a cytosine; N, any nucleotide.

In addition, notation “N(3-5)” means that indicated consensus position may have 3 to 5 any (N) nucleotides. For example, a consensus sequence “AWN(4-6)” represents 3 possible variants—with 4, 5, or 6 any nucleotides at the end: AWNNNN, AWNNNNN, AWNNNNNN.

The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:
DNA-DNA hybrids (Meinkoth and Wahl, Anal. Biochem., 138: 267-284, 1984):
   Tₘ=81.5° C.+16.6×log[Na⁺]^{a}+0.41×%[G/C^{b}]−500×[L^{c}]⁻¹−0.61×% formamide
DNA-RNA or RNA-RNA hybrids:
   Tₘ=79.8+18.5 (log₁₀[Na⁺]^{a})+0.58 (% G/C^{b})+11.8 (% G/C^{b})²−820/L^{c}
oligo-DNA or oligo-RNA^{d} hybrids:
   For <20 nucleotides: Tₘ=2 (Iₙ)
   For 20-35 nucleotides: Tₘ=22+1.46 (Iₙ)
^{a} or for other monovalent cation, but only accurate in the 0.01-0.4 M range.
^{b} only accurate for % GC in the 30% to 75% range.
^{c} L=length of duplex in base pairs.
^{d} Oligo, oligonucleotide; Iₙ, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

“Recombinant” (or transgenic) with regard to a cell or an organism means that the cell or organism contains an exogenous polynucleotide which is introduced by gene technology and with regard to a polynucleotide means all those constructions brought about by gene technology/recombinant DNA techniques in which either

(a) the sequence of the polynucleotide or a part thereof, or

(b) one or more genetic control sequences which are operably linked with the polynucleotide,

for example a promoter, or

(c) both a) and b)

are not located in their wildtype genetic environment or have been modified.

It shall further be noted that the term “isolated nucleic acid” or “isolated polypeptide” may in some instances be considered as a synonym for a “recombinant nucleic acid” or a “recombinant polypeptide”, respectively and refers to a nucleic acid or polypeptide that is not located in its natural genetic environment or cellular environment, respectively, and/or that has been modified by recombinant methods. An isolated nucleic acid sequence or isolated nucleic acid molecule is one that is not in its native surrounding or its native nucleic acid neighbourhood, yet it is physically and functionally connected to other nucleic acid sequences or nucleic acid molecules and is found as part of a nucleic acid construct, vector sequence or chromosome. Typically, the isolated nucleic acid is obtained by isolating RNA from cells under laboratory conditions and converting it in copyDNA (cDNA).

The term “control sequence” is defined herein to include all sequences affecting for the expression of a polynucleotide, including but not limited thereto, the expression of a polynucleotide encoding a polypeptide. Each control sequence may be native or foreign to the polynucleotide or native or foreign to each other. Such control sequences include, but are not limited to, a leader, polyadenylation sequence, propeptide sequence, promoter, 5′-UTR, ribosomal binding site (RBS, shine dalgarno sequence), 3′-UTR, signal peptide sequence, and transcription terminator. At a minimum, the control sequence includes a promoter and transcriptional start and stop signals. The term “operably linked” means that the described components are in a relationship permitting them to function in their intended manner. For example, a regulatory sequence operably linked to a coding sequence is ligated in such a way that expression of the coding sequence is achieved under condition compatible with the control sequences.

“Parent” (or “reference” or “template”) of a nucleic acid, protein, enzyme, or organism (also called “parent nucleic acid”, “reference nucleic acid”, “template nucleic acid”, “parent protein” “reference protein”, “template protein”, “parent enzyme” “reference enzyme”, “template enzyme”, “parent organism” “reference organism”, or “template organism”)) is the starting point for the introduction of changes (e.g. by introducing one or more nucleic acid or amino acid substitutions) resulting in “variants” of the parent. Thus, terms such as “enzyme variant” or “sequence variant” or “variant protein” are used to distinguish the modified or variant sequences, proteins, enzymes, or organisms from the parent sequences, proteins, enzymes, or organisms that are the origin for the respective variant sequences, proteins, enzymes, or organisms. Therefore, parent sequences, proteins, enzymes, or organisms include wild type sequences, proteins, enzymes, or organisms, and variants of wild-type sequences, proteins, enzymes, or organisms which are used for development of further variants. Variant proteins or enzymes differ from parent proteins or enzymes in their amino acid sequence to a certain extent; however, variants at least maintain the functional properties, e.g., enzyme properties, of the respective parent. In one embodiment, enzyme properties are improved in variant enzymes when compared to the respective parent enzyme. In one embodiment, variant enzymes have at least the same enzymatic activity when compared to the respective parent enzyme or variant enzymes have increased enzymatic activity when compared to the respective parent enzyme.

In describing the variants, the nomenclature described as follows is used: Abbreviations for single amino acids used within this invention are according to the accepted IUPAC single letter or three letter amino acid abbreviation. While the definitions below describe variants in the context of amino acid changes, nucleic acids may be similarly modified, e.g. by substitutions, deletions, and/or insertions of nucleotides.

“Substitutions” are described by providing the original amino acid followed by the number of the position within the amino acid sequence, followed by the substituted amino acid. For example, the substitution of histidine at position 120 with alanine is designated as “His120Ala” or “H120A”.

“Deletions” are described by providing the original amino acid followed by the number of the position within the amino acid sequence, followed by *. Accordingly, the deletion of glycine at position 150 is designated as “Gly150*” or G150*”. Alternatively, deletions are indicated by e.g. “deletion of D183 and G184”.

“Insertions” are described by providing the original amino acid followed by the number of the position within the amino acid sequence, followed by the original amino acid and the additional amino acid. For example, an insertion at position 180 of lysine next to glycine is designated as

“Gly180GlyLys” or “G180GK”. When more than one amino acid residue is inserted, such as e.g. a Lys and Ala after Gly180 this may be indicated as: Gly180GlyLysAla or G180GKA.

In cases where a substitution and an insertion occur at the same position, this may be indicated as S99SD+S99A or in short S99AD.

In cases where an amino acid residue identical to the existing amino acid residue is inserted, it is clear that degeneracy in the nomenclature arises. If for example a glycine is inserted after the glycine in the above example this would be indicated by G180GG.

Variants comprising multiple alterations are separated by “+”, e.g. “Arg170Tyr+Gly195Glu” or “R170Y+G195E” representing a substitution of arginine and glycine at positions 170 and 195 with tyrosine and glutamic acid, respectively. Alternatively, multiple alterations may be separated by space or a comma e.g. R170Y G195E or R170Y, G195E respectively.

Where different alterations can be introduced at a position, the different alterations are separated by a comma, e.g. “Arg170Tyr, Glu” represents a substitution of arginine at position 170 with tyrosine or glutamic acid. Alternatively, different alterations or optional substitutions may be indicated in brackets e.g. Arg170[Tyr, Gly] or Arg170{Tyr, Gly} or in short R170 [Y,G] or R170 {Y, G}.

Variants may include one or more alterations, either of the same type, e.g., all substitutions, or combinations of substitutions, deletions, and/or insertions. Alterations can be introduced to the nucleic acid or to the amino acid sequence.

In one embodiment, the variants of de-regulated adenylate cyclase includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or more alterations and has adenylate cyclase activity.

Variants of the de-regulated adenylate cyclase sequences include nucleic acids and polypeptides having about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to any of SEQ ID NO: 1 to 10 or 10 to 20, respectively, and having adenylate cyclase activity, and preferably without or with an inactive or downregulated or absent regulatory part of the wildtype adenylate cyclase.

For substituting amino acids of a base sequence selected from any of the sequences SEQ ID NO. 1 to 10 or 26 without regard to the occurrence of amino acids in other of these sequences, the following applies, wherein letters indicate L amino acids using their common abbreviation and bracketed numbers indicate preference of replacement (higher numbers indicate higher preference): A may be replaced by any amino acid selected from S (1), C(0), G (0), T (0) or V (0). C may be replaced by A (0). D may be replaced by any amino acid selected from E (2), N (1), Q (0) or S(0). E may be replaced by any amino acid selected from D (2), Q (2), K (1), H (0), N(0), R (0) or S(0). F may be replaced by any amino acid selected from Y (3), W (1), I (0), L (0) or M (0). G may be replaced by any amino acid selected from A (0), N(0) or S(0). H may be replaced by any amino acid selected from Y (2), N (1), E (0), Q (0) or R (0). I may be replaced by any amino acid selected from V (3), L (2), M (1) or F (0). K may be replaced by any amino acid selected from R (2), E (1), Q (1), N(0) or S(0). L may be replaced by any amino acid selected from I (2), M (2), V (1) or F (0). M may be replaced by any amino acid selected from L (2), I (1), V (1), F (0) or Q (0). N may be replaced by any amino acid selected from D (1), H (1), S (1), E (0), G (0), K (0), Q (0), R (0) or T (0). Q may be replaced by any amino acid selected from E (2), K (1), R (1), D (0), H (0), M (0), N(0) or S(0). R may be replaced by any amino acid selected from K (2), Q (1), E (0), H (0) or N(0). S may be replaced by any amino acid selected from A (1), N (1), T (1), D (0), E (0), G (0), K (0) or Q (0). T may be replaced by any amino acid selected from S (1), A (0), N(0) or V (0). V may be replaced by any amino acid selected from I (3), L (1), M (1), A (0) or T (0). W may be replaced by any amino acid selected from Y (2) or F (1). Y may be replaced by any amino acid selected from F (3), H (2) or W (2).

Nucleic adds and polypeptides may be modified to include tags or domains. Tags may be utilized for a variety of purposes, including for detection, purification, solubilization, or immobilization, and may include, for example, biotin, a fluorophore, an epitope, a mating factor, or a regulatory sequence. Domains may be of any size and which provides a desired function (e.g., imparts increased stability, solubility, activity, simplifies purification) and may include, for example, a binding domain, a signal sequence, a promoter sequence, a regulatory sequence, an N-terminal extension, or a C30 terminal extension. Combinations of tags and/or domains may also be utilized.

The term “fusion protein” refers to two or more polypeptides joined together by any means known in the art. These means include chemical synthesis or splicing the encoding nucleic acids by recombinant engineering.

Gene Editing

Gene editing or genome editing is a type of genetic engineering in which DNA is inserted, replaced, or removed from a genome and which can be obtained by using a variety of techniques such as “gene shuffling” or “directed evolution” consisting of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547), or with “T-DNA activation” tagging (Hayashi et al. Science (1992) 1350-1353), where the resulting transgenic organisms show dominant phenotypes due to modified expression of genes close to the introduced promoter, or with “TILLING” (Targeted Induced Local Lesions In Genomes) and refers to a mutagenesis technology useful to generate and/or identify nucleic acids encoding proteins with modified expression and/or activity. TILLING also allows selection of organisms carrying such mutant variants. Methods for TILLING are well known in the art (McCallum et al., (2000) Nat Biotechnol 18: 455-457; reviewed by Stemple (2004) Nat Rev Genet 5(2): 145-50). Another technique uses artificially engineered nucleases like Zinc finger nucleases, Transcription Activator-Like Effector Nucleases (TALENs), the CRISPR/Cas system, and engineered meganuclease such as re-engineered homing endonucleases (Esvelt, K M.; Wang, H H. (2013), Mol Syst Biol 9 (1): 641; Tan, W S. et al. (2012), Adv Genet 80: 37-97; Puchta, H.; Fauser, F. (2013), Int. J. Dev. Biol 57: 629-637).

“Enzymatic activity” means at least one catalytic effect exerted by an enzyme. In one embodiment, enzymatic activity is expressed as units per milligram of enzyme (specific activity) or molecules of substrate transformed per minute per molecule of enzyme (molecular activity). In the case of adenylate cyclase activity, the molecular enzyme activity can be understood as the number of cAMP molecules produced per minute per molecule of adenylate cyclase or adenylate cyclase containing part of a protein.

Alignment of sequences is preferably done with the algorithm of Needleman and Wunsch Needleman and Wunsch algorithm—Needleman, Saul B. & Wunsch, Christian D. (1970). “A general method applicable to the search for similarities in the amino acid sequence of two proteins”. Journal of Molecular Biology. 48 (3): 443-453. This algorithm is, for example, implemented into the “NEEDLE” program, which performs a global alignment of two sequences. The NEEDLE program, is contained within, for example, the European Molecular Biology Open Software Suite (EMBOSS), a collection of various programs: The European Molecular Biology Open Software Suite (EMBOSS), Trends in Genetics 16 (6), 276 (2000).

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning the two sequences, in a second step, an identity value shall be determined from the alignment. Therefore, according to the present description the following calculation of percent-identity applies:

%-identity=(identical residues/length of the alignment region which is showing the shorter sequence over its complete length)*100. Thus, sequence identity in relation to comparison of two amino acid sequences according to this embodiment is calculated by dividing the number of identical residues by the length of the alignment region which is showing the shorter sequence over its complete length. This value is multiplied with 100 to give “%-identity”. According to the example provided above, %-identity is: (6/8)*100=75%.

Gene Editing

A number of techniques for targeted modification in a genome of an organism are known. Most widely known is the technology known as CRIPR or CRISPR/CAS:

The CRISPR (clustered regularly interspaced short palindromic repeats) technology may be used to modify the genome of a target organism, for example to introduce any given DNA fragment into nearly any site of the genome, to replace parts of the genome with desired sequences or to precisely delete a given region in the genome of a target organism. This allows for unprecedented precision of genome manipulation.

The CRISPR system was initially identified as an adaptive defense mechanisms of bacteria belonging to the genus of Streptococcus (WO2007/025097). Those bacterial CRISPR systems rely on guide RNA (gRNA) in complex with cleaving proteins to direct degradation of complementary sequences present within invading viral DNA. The application of CRISPR systems for genetic manipulation in various eukaryotic organisms have been shown (WO2013/141680; WO2013/176772; WO2014/093595). Cas9, the first identified protein of the CRISPR/Cas system, is a large monomeric DNA nuclease guided to a DNA target sequence adjacent to the PAM (protospacer adjacent motif) sequence motif by a complex of two noncoding RNAs: CRSIPR RNA (crRNA) and trans-activating crRNA (tracrRNA). Also a synthetic RNA chimera (single guide RNA or sgRNA) created by fusing crRNA with tracrRNA was shown to be equally functional (WO2013/176772). CRISPR systems from other sources comprising DNA nucleases distinct from Cas9 such as Cpf1, C2c1p or C2c3p have been described having the same functionality (WO2016/0205711, WO2016/205749). Other authors describe systems in which the nuclease is guided by a DNA molecule instead of an RNA molecule. Such system is for example the AGO system as disclosed in US2016/0046963.

Several research groups have found that the CRISPR cutting properties could be used to disrupt target regions in almost any organism's genome with unprecedented ease. Recently it became clear that providing a template for repair allows for editing the genome with nearly any desired sequence at nearly any site, transforming CRISPR into a powerful gene editing tool (WO2014/150624, WO2014/204728). The template for repair is addressed as donor nucleic acid comprising at the 3′ and 5′ end sequences complementary to the target region allowing for homologous recombination in the respective template after introduction of doublestrand breaks in the target nucleic acid by the respective nuclease.

The main limitation in choosing the target region in a given genome is the necessity of the presence of a PAM sequence motif close to the region where the CRISPR related nuclease introduces doublestrand breaks. However, various CRISPR systems recognize different PAM sequence motifs. This allows choosing the most suitable CRISPR system for a respective target region. Moreover, the AGO system does not require a PAM sequence motif at all.

The technology may for example be applied for alteration of gene expression in any organism, for example by exchanging the promoter upstream of a target gene with a promoter of different strength or specificity. Other methods disclosed in the prior art describe the fusion of activating or repressing transcription factors to a nuclease minus CRISPR nuclease protein. Such fusion proteins may be expressed in a target organism together with one or more guide nucleic acids guiding the transcription factor moiety of the fusion protein to any desired promoter in the target organism (WO2014/099744; WO2014/099750). Knockouts of genes may easily be achieved by introducing point mutations or deletions into the respective target gene, for example by inducing non-homologous-end-joining (NHEJ) which usually leads to gene disruption (WO2013/176772).

Recombinant Organism

The term “recombinant organism” refers to a eukaryotic organism (yeast, fungus, alga, plant, animal) or to a prokaryotic microorganism (e.g., bacteria) which has been genetically altered, modified or engineered such that it exhibits an altered, modified or different genotype as compared to the wild-type organism which it was derived from. Preferably, the “recombinant organism” comprises an exogenous nucleic acid. “Recombinant organism”, “genetically modified organism” and “transgenic organism” are used herein interchangeably. The exogenous nucleic acid can be located on an extrachromosomal piece of DNA (such as plasmids) or can be integrated in the chromosomal DNA of the organism. Recombinant is understood as meaning that the nucleic acid(s) used are not present in, or originating from, the genome of said organism, or are present in the genome of said organism but not at their natural locus in the genome of said organism, it being possible for the nucleic acids to be expressed under the control of one or more endogenous and/or exogenous control element.

“Host Cells”

Host cells also called host organisms may be any cell selected from bacterial cells, yeast cells, fungal, algal or cyanobacterial cells, non-human animal or mammalian cells, or plant cells. The skilled artisan is well aware of the genetic elements that must be present on the genetic construct to successfully transform, select and propagate host cells containing the sequence of interest.

In one embodiment host cell or host organisms are used interchangeably.

Typical host cells are Bacteria, such as gram positive: Bacillus, Streptomyces. Useful gram positive bacteria include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus iautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus. Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

Further typical host cells are gram negative: E. coli, Pseudomonas, preferred gram negative bacteria are Escherichia coli and Pseudomonas sp., preferably, Pseudomonas purrocinia (ATCC 15958) or Pseudomonas fluorescens (NRRL B-11).

Further typical host cells are fungi, such as Aspergillus, Fusarium, Trichoderma. The microorganism may be a fungal cell. “Fungi” as used herein includes the phyla Ascomycota, Basidiomycota, Chytridiomycota, and Zygomycota as weil as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g. Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor. Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucana (IFO 6113), Verticillum alboatrum, Verticillum dahlie, Arthromyces ramosus (FERM P-7754), Caldariomyces fumago, Ulocladium chartarum, Embellisia alli or Dreschlera halodes.

Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A).

Further preferred fungi include strains belonging to the subdivision Zygomycotina, class Mycoraceae, e.g. Rhizopus or Mucor, in particular Mucor hiemalis.

Further typical host cells are yeasts. Such as Pichia species or Saccharomyces species. The fungal host cell may be a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g. genera Kluyveromyces, Pichia, and Saccharomyces). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sporobolomyces and Bullera) and Cryptococcaceae (e.g. genus Candida). Also typical host cells are Eukaryotes such as non-human animal, non-human mammal, avian, reptilian, insect, plant, yeast, fungi or plants.

Preferably the host organism according to the invention can be a gram positive or gram negative prokaryotic microorganism.

Useful gram positive prokaryotic microorganism include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus Jautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus. Some other preferred bac-teria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium.

Other pre-ferred bacteria include Rhodobacter sphaeroides, Rhodomonas palustri, Streptococcus lactis. Further preferred bacteria include strains belonging to Myxococcus, e.g., M. virescens.

Further typical prokaryotic organisms are gram negative: Escherichia coli, Pseudomonas, preferred gram negative prokaryotic microorganisms are Escherichia coli and Pseudomonas sp., preferably, Pseudomonas purrocinia (ATCC 15958) or Pseudomonas fluorescens (NRRL B-11).

Most preferably the prokaryotic microorganism is Escherichia coli.

The term “monosaccharide” preferably means a sugar of 5-9 carbon atoms that is an aldose (e.g. D-glucose, D-galactose, D-mannose, D-ribose, D-arabinose, L-arabinose, D-xylose, etc.), a ketose (e.g. D-fructose, D-sorbose, D-tagatose, etc.), a deoxysugar (e.g. L-rhamnose, L-fucose, etc.), a deoxyaminosugar (e.g. N-acetylglucosamine, N-acetylmannosamine, N-acetylgalactosamine, etc.), an uronic acid, a ketoaldonic acid (e.g. sialic acid) or equivalents.

The term “oligosaccharide” preferably means a sugar polymer containing at least three monosaccharide units (vide supra). The oligosaccharide can have a linear or branched structure containing monosaccharide units that are linked to each other by interglycosidic linkage. Examples are without limitation maltodextrins, cellodextrins, human milk oligosaccharide, fructooligosacharides and galactooligosaccharides.

Preferably the oligosaccharide is a human milk oligosaccharide (HMO).

The term “human milk oligosaccharide” or “HMO” preferably means a complex carbohydrate found in human breast milk (Urashima et al.: Milk Oligosaccharides. Nova Science Publishers, 2011). The HMOs have a core structure being a lactose unit at the reducing end that can be elongated by one or more β-N-acetyl-lactosaminyl and/or one or more β-lacto-N-biosyl units, and which core structures can be substituted by an α L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure.

The non-acidic (or neutral) HMOs can be fucosylated or non-fucosylated. Examples of such neutral non-fucosylated HMOs include lacto-N-triose (LNTri, GIcNAc(β1-3)Gal(β1-4)Glc), lactoN-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaosel (LNFP-1), lactoN-difucohexaose I (LNDFH-I), 3-fucosyllactose (3′-FL), difucosyllactose (2,3-DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT). Examples for human milk oligosacchardides can also be found in Niñonuevo M R et al. (2006). J. Agric. Food Chem. 54:7471-7480, Bode L (2009) Nutr. Rev. 67:183-191, Bode L (2012) Glyco-biology 22:1147-1162, Bode L (2015) Early Hum. Dev. 91:619-622

More preferably the HMO is a neutral or acidic HMO.

Even more preferably the oligosaccharide is 2′-fucosyllactose (2′-FL), 6′-sialyllactose (6′-SL) and/or lacto-N-tetraose (LNT).

The terms “increase”, “improve” or “enhance” in the context of enzyme activity or amounts of cAMP or fine chemical production, carbon conversion efficiency, space-time-yield or growth or carbon source flexibility are interchangeable and shall mean in the sense of the application at least a 3%, 4%, 5%, 6%, 7%, 8%, 9% or 10%, preferably at least 15% or 20%, more preferably 25%, 30%, 35% or 40% or more increase in comparison to the controls such as but not limited to the non-modified host organism.

The terms “decrease”, “reduced” or “lowered” in the context of gene expression or protein presence or protein abundance or inactivation are interchangeable and shall mean in the sense of the application at least a 3%, 4%, 5%, 6%, 7%, 8%, 9% or 10%, preferably at least 15% or 20%, more preferably 25%, 30%, 35% 40%, 50%, 60%, 70%, 80%, 85%, 90%, 92%, 94%, 95% or 98% or greater reduction in comparison to the controls as defined herein.

The term “enhanced production of oligosaccharides” refers to enhanced productivity of oligosaccharides and/or an enhanced titer of oligosaccharides and/or an enhanced carbon conversion efficiency rate compared to its parent strain. The production of oligosaccharides by the microorganism in the culture medium can be recorded unambiguously by standard analytical means known by those skilled in the art. Some genetically modified microorganisms with enhanced production of oligosaccharides (e.g. HMOs) are disclosed in patent applications published as WO 2016/008602, WO2013/182206, EP2379708, U.S. Pat. No. 9,944,965, WO2012/112777, WO2001/04341 and US2005019874 for E. coli strains. All of these disclosures are herein incorporated by reference.

Furthermore, the inventors found that surprisingly the carbon conversion efficiency, carbon substrate flexibility and space/time of the production of oligosaccharides by a prokaryotic organism can be increased by manipulating the PTS system in a way that prevents Crr protein, or proteins of said prokaryotic organism corresponding to the Crr protein, in participating in the PTS either by decreasing or preventing the expression of the crr gene ((SEQ ID NO: 25) or variants thereof, or by inactivation or reduction of the Crr protein (SEQ ID NO: 26) or variants thereof. Host organism harbouring such inactivated or reduced proteins of the Crr family or decreased or prevented expression of the genes of the crr gene family are in one embodiment prokaryotic microorganism.

In one aspect of the invention, increased carbon substrate flexibility is the characteristic of a modified microorganism to grow on a carbon source that the unmodified microorganism is unable to grow on or to grow substantially better on a carbon source than the control, which maybe a wildtype cell or genetically modified microorganism without an alteration in respect to the adenylate cyclase activity and/or an alteration in respect to a gene or protein corresponding to the crr gene (SEQ ID NO: 25) or Crr protein (SEQ ID NO: 26), respectively.

In one embodiment the methods of the invention are methods for the increase of space-timeyield of one or more fine chemicals, preferably one or more oligosaccharides, produced by a genetically modified microorganism and/or for the increase of carbon substrate flexibility and/or the carbon-conversion-efficiency of the production of one or more fine chemicals, preferably one or more oligosaccharides, by a genetically modified microorganism compared to the microorganism without alterations concerning gene or protein that correspond to the crr gene (SEQ ID NO: 25) or Crr protein (SEQ ID NO: 26), respectively, including the steps of providing a microorganism capable of producing the one or more fine chemicals, increasing the Adenosine 3′,5′-cyclic monophosphate (cAMP, CAS Number: 60-92-4) levels of the microorganism by inactivation or absence of the Crr protein or the endogenous protein corresponding to the Crr protein in E. coli (SEQ ID NO: 26), maintaining said altered microorganism in a setting allowing it to grow, growing the altered microorganism in the presence of substrates and nutrients and under conditions suitable for the production of one or more fine chemicals and optionally separating one or more fine chemicals from the altered microorganism or remainder thereof. In one embodiment the altered microorganism is suitable to produce said one or more fine chemicals in the non-modified and the modified form.

In one embodiment, the variant CRR proteins includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or more alterations compared to the unmodified Crr protein or protein corresponding to the Crr protein, and the abundance, activity and/or lifetime of the variant is reduced compared to the unmodified CRR protein family member of that microorganism.

Variants include nucleic acids and polypeptides having about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 25 or 26, respectively.

The term “Genetically modified microorganism” refers to a prokaryotic microorganism (e.g., bacteria) which has been genetically altered, modified or engineered such that it exhibits an altered, modified or different genotype as compared to the wild-type organism which it was derived from. “Genetically modified microorganism”, “recombinant microorganism” and “transgenic microorganism” are used herein interchangeably. The exogenous nucleic acid in said genetically modified microorganisms can be located on an extrachromosomal piece of DNA (such as plasmids) or can be integrated in the chromosomal DNA of the organism.

The genetically modified microorganism according to the invention can be a gram positive or gram-negative prokaryotic microorganism.

Gram positive prokaryotic microorganism useful to generate the genetically modified microorganisms of the invention and those useful in the inventive methods include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus iautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus. Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

Further typical prokaryotic organisms useful to generate the genetically modified microorganisms of the invention and those useful in the inventive methods are gram negative: Escherichia coli, Pseudomonas, preferred gram negative prokaryotic microorganisms are Escherichia coli and Pseudomonas sp., preferably, Pseudomonas purrocinia (ATCC 15958) or Pseudomonas fluorescens (NRRL B-11).

Most preferably the prokaryotic microorganism useful to generate the genetically modified microorganisms of the invention and those useful in the inventive methods is Escherichia coli.

The PTS carbohydrate utilization system (PTS) is a well characterized carbohydrate transport system utilized by microorganisms such as bacteria. See Postma et al. 1993 (Postma P W, Lengeler J W, Jacobson G R. Phosphoenolpyruvate: carbohydrate phosphotransferase systems of bacteria. Microbiol Rev. 1993 September; 57(3): 543-94.) and Tchieu et al. 2001 (Tchieu J H, Norris V, Edwards J S, Saier M H Jr. The complete phosphotransferase system in Escherichia coli. J Mol Microbiol Biotechno. 2001 July; 3(3):329-46), which are incorporated herein by reference in their entirely. Exemplary bacteria comprising the PTS include those from the genera Bacillus, Clostridium, Enterobacteriaceae, Enterococcus, Erwinia, Escherichia, Klebsiella, Lactobacillus, Lactococcus, Mycoplasma, Pasteurella, Rhodobacter, Rhodoseudomonas, Salmonella, Staphylococcus, Streptococcus, Vibrio, and Xanthomonas. Exemplary species include E. coli, Salmonella typhimurium, Staphylococcus camosus, Bacillus subtilis, Mycoplasma capricolum, Enterococcus faecalis, Staphylococcus aureus, Streptococcus salivarius, Streptococcus mutans, Klebsiella pneumoniae, Staphylococcus camosus, Streptococcus sanguis, Rhodobacter capsulatus, Vibrio alginolyticus, Erwinia chrysanthemi, Xanthomonas campestris, Lactococcus lactis, Lactobacillus casei, Rhodoseudomonas sphaeroides, Erwinia carotovora, Pasteurella multocida, and Clostridium acetobutylicum.

Surprisingly, the inventors have for the first time that a reduction in Crr protein abundance results in an increased space-time-yield, carbon substrate flexibility or carbon-conversion-efficiency of oligosaccharides produced by modified microorganism, preferably genetically modified microorganism.

The modified microorganism, preferably genetically modified microorganism, with microorganism, with reduced or absent Crr protein abundance can be achieved by a number of means, such as reducing the crr gene expression including knock-outs of the gene, or deletions in part or full, antisense or RNAi approaches, or other recombinant methods for example gene editing methods like CRISPR/CAS, or even segregation of the Crr protein by an unusual binding partner, e.g. antibodies.

In one embodiment the manipulation, preferably reduction in level of or complete removal of the Crr protein is done in an inducible manner and the increase in the space-time-yield, carbon substrate flexibility and/or carbon-conversion-efficiency is compared to the genetically modified microorganisms without such induction. Methods for the inducer dependent gene expression for example by the inducer Isopropyl β-d-1-thiogalactopyranoside (IPTG) are known in the art.

In a preferred embodiment the methods of the invention are methods for the increase of spacetime-yield of one or more fine chemicals produced by a microorganism as well as for the increase of carbon substrate flexibility and the carbon-conversion-efficiency of the production of one or more fine chemicals by a microorganism including the steps of providing a microorganism capable of producing the one or more fine chemicals, inactivating or downregulating in the microorganism the locus of a gene corresponding to SEQ ID NO: 25 or variants thereof, or inactivating or removing the protein corresponding to the Crr protein as encoded by SEQ ID NO: 25 or variants thereof, maintaining said genetically modified microorganism in a setting allowing it to grow, growing said genetically modified microorganism in the presence of substrates and nutrients and under conditions suitable for the production of one or more fine chemicals and optionally separating one or more fine chemicals from the genetically modified microorganism or remainder thereof.

The activity of the Crr protein, variants thereof or proteins corresponding to the Crr protein in a microorganism is to be understood as the normal biological function of the Crr protein or variants thereof or proteins corresponding to the Crr protein. This can involve for example kinase activity since the Crr protein is known to comprise a kinase domain. Inactivation is to be understood in that said activity is not present to at the same normal level, but substantially lower or entirely absent. The abundance of these proteins of interest at normal levels is required for the normal biological function as well. If the abundance of said proteins of interest is reduced substantially, the biological function and hence overall activity will be reduced. If the proteins of interest are absent, e.g. since the gene encoding it has been made non-functional, has been deleted in part or full, has been knocked-out or its expression is prevented, the biological function is sooner or later abolished.

In a preferred aspect of the invention, the host cell useful in the methods and uses of the invention carries the deregulated adenylate cyclase of the invention in combination with the decreased expression of the crr gene or variant thereof and/or an inactivation of or reduction of the Crr protein or variants thereof on the carbon conversion efficiency, carbon substrate flexibility and space/time of the production of oligosaccharides by a prokaryotic organism.

In one embodiment the methods of the invention include a step of inactivating or removing in the genetically modified microorganism the Crr protein or the endogenous protein(s) corresponding to the Crr protein in E. coli (SEQ ID NO: 26) as defined herein before the growth of the genetically modified microorganism. The inactivation or removal of the CRR protein family member can be performed before, at the same time or after the deregulated adenylate cyclase is present for the first time in the microorganism, i.e. before, at the same time or after any of the following actions is performed:
a. Inactivating the regulatory activity found in a wildtype adenylate cyclase in the host organism, and/or
b. generating in the host organism a mutated adenylate cyclase lacking the regulatory activity found in a wildtype adenylate cyclase, and/or
c. introduction into the host organism of a mutated adenylate cyclase lacking the regulatory activity found in a wildtype adenylate cyclase

Another preferred embodiment of the invention is a composition comprising one or more types of host cells comprising a deregulated adenylate cyclase and/or the abundance and/or activity of the Crr protein (SEQ ID NO: 26), of variants thereof or of endogenous protein corresponding to the Crr protein in one or more microorganisms is decreased compared to a control host cell, i.e. a host cell with the wildtype adenylate cyclase and/or wildtype level and activity of the Crr protein (SEQ ID NO: 26), of variants thereof or of endogenous protein corresponding to the Crr protein in said microorganism. In a more preferred embodiment, the composition of the invention further comprises one or more fine chemicals, preferably one or more human milk oligosaccharides.

Preferably, the host cell or genetically modified microorganism producing 2′-fucosyllactose (2′-FL) of the invention and useful in the methods of the invention is an Escherichia coli strain and comprises at least:
a 1,2-fucosyltransferase enzyme, and
the means to provide fucose moieties and lactose to the fucosyltransferase enzyme suitable for the production of 2′-FL

Preferably, the host cell or genetically modified microorganism producing 6′-sialyllactose (6′-SL) of the invention and useful in the methods of the invention is an Escherichia coli strain and comprises at least:
a sialyltransferase enzyme, and
the means to provide sialic acid moieties and lactose to the sialyltransferase enzyme suitable for the production of 6′-SL

Preferably, the host cell or genetically modified microorganism producing lacto-N-tetraose (LNT) of the invention and useful in the methods of the invention is an Escherichia coli strain and comprises at least:
a β 1,3-Glactosyltransferase enzyme, and
the means to provide nucleotide activated galactose and LNT2 to the β 1,3-Glactosyltransferase enzyme suitable for the production of LNT

Culturing a host cell or microorganism frequently requires that cells be cultured in a medium containing various nutrition sources, like a carbon source, nitrogen source, and other nutrients, including but not limited to amino acids, vitamins, minerals, required for growth of those cells. The fermentation medium may be a minimal medium as described in, e.g., WO 98/37179, or the fermentation medium may be a complex medium comprising complex nitrogen and carbon sources, wherein the complex nitrogen source may be partially hydrolyzed as described in WO 2004/003216.

Thus, fermentation medium comprises components required for the growth of the cultivated microorganism or host cell. In one embodiment, the fermentation medium comprises one or more components selected from the group consisting of nitrogen source, phosphor source, sulfur source and salt, and optionally one or more further components selected the group consisting of micronutrients, like vitamins, amino acids, minerals, and trace elements. In one embodiment, the fermentation medium also comprises a carbon source. Such components are generally well known in the art (see, e.g., Ausubel, et al, Short Protocols in Molecular Biology, 3rd ed., Wiley & Sons, 1995; Sambrook, et al., Molecular Cloning: A Laboratory Manual, Second Edition, 1989 Cold Spring Harbor, N.Y.; Talbot, Molecular and Cellular Biology of Filamentous Fungi: A Practical Approach, Oxford University Press, 2001; Kinghom and Turner, Applied Molecular Genetics of Filamentous Fungi, Cambridge University Press, 1992; and Bacillus (Biotechnology Handbooks) by Colin R. Harwood, Plenum Press, 1989). Culture conditions for a given cell type may also be found in the scientific literature and/or from the source of the cell such as the American Type Culture Collection (ATCC) and Fungal Genetics Stock Center.

As sources of nitrogen, inorganic and organic nitrogen compounds may be used, both individually and in combination. Suitable organic nitrogen sources include but are not limited to proteincontaining substances, such as an extract from microbial, animal or plant cells, including but not limited thereto plant protein preparations, soy meal, corn meal, pea meal, corn gluten, cotton meal, peanut meal, potato meal, meat and casein, gelatines, whey, fish meal, yeast protein, yeast extract, tryptone, peptone, bacto-tryptone, bacto-peptone, wastes from the processing of microbial cells, plants, meat or animal bodies, and combinations thereof. Inorganic nitrogen sources include but are not limited to ammonium, nitrate, and nitrite, and combinations thereof. In one embodiment, the fermentation medium comprises a nitrogen source, wherein the nitrogen source is a complex or a defined nitrogen source or a combination thereof. In one embodiment, the complex nitrogen source is selected from the group consisting of plant protein, including but not limited to, potato protein, soy protein, corn protein, peanut, cotton protein, and/or pea protein, casein, tryptone, peptone and yeast extract and combinations thereof. In one embodiment, the defined nitrogen source is selected from the group consisting of ammonia, ammonium, ammonium salts, (e.g., ammonium chloride, ammonium nitrate, ammonium phosphate, ammonium sulfate, ammonium acetate), urea, nitrate, nitrate salts, nitrite, and amino acids, including but not limited to glutamate, and combinations thereof.

In one embodiment, the fermentation medium further comprises at least one carbon source. The carbon source can be a complex or a defined carbon source or a combination thereof. Various sugars and sugar-containing substances are suitable sources of carbon, and the sugars may be present in different stages of polymerisation. The complex carbon sources include, but are not limited thereto, molasse, corn steep liquor, cane sugar, dextrin, starch, starch hydrolysate, and cellulose hydrolysate, and combinations thereof. The defined carbon sources include, but are not limited thereto, carbohydrates, organic acids, and alcohols. In one embodiment, the defined carbon sources include, but are not limited thereto, glucose, fructose, galactose, xylose, arabinose, sucrose, maltose, lactose, gluconate, acetic acid, propionic acid, lactic acid, formic acid, malic acid, citric acid, fumaric acid, glycerol, inositol, mannitol and sorbitol, and combinations thereof. In one embodiment, the defined carbon source is provided in form of a syrup, which can comprise up to 20%, up to 10%, or up to 5% impurities. In one embodiment, the carbon source is sugar beet syrup, sugar cane syrup, corn syrup, including but not limited to, high fructose corn syrup. The complex carbon source includes, but is not limited to, molasses, corn steep liquor, dextrin, and starch, or combinations thereof. In a preferred embodiment defined carbon source includes, but is not limited to, glucose, fructose, galactose, xylose, arabinose, sucrose, maltose, dextrin, lactose, gluconate or combinations thereof.

In another preferred embodiment, one carbon source or the carbon source is sucrose, and with this carbon source the method of the invention and the host cell or genetically modified microorganism of the invention offer even a greater advantage compared to the organisms and the methods known in the art.

In one embodiment, the fermentation medium also comprises a phosphor source, including, but not limited to, phosphate salts, and/or a sulphur source, including, but not limited to, sulphate salts. In one embodiment, the fermentation medium also comprises a salt. In one embodiment, the fermentation medium comprises one or more inorganic salts, including, but not limited to alkali metal salts, alkali earth metal salts, phosphate salts and sulphate salts. In one embodiment, the one or more salt includes, but is not limited to, NaCl, KH2PO4, MgSO4, CaCl2), FeCl3, MgCl2, MnCl2, ZnSO4, Na2MoO4 and CuSO4. In one embodiment, the fermentation medium also comprises one or more vitamins, including, but not limited to, thiamine chloride, biotin, vitamin B12. In one embodiment, the fermentation medium also comprises trace elements, including, but not limited to, Fe, Mg, Mn, Co, and Ni. In one embodiment, the fermentation medium comprises one or more salt cations selected from the group consisting of Na, K, Ca, Mg, Mn, Fe, Co, Cu, and Ni. In one embodiment, the fermentation medium comprises one or more divalent or trivalent cations, including but not limited to, Ca and Mg.

In one embodiment, the fermentation medium also comprises an antifoam.

In one embodiment, the fermentation medium also comprises a selection agent, including, but not limited to, an antibiotic, including, but not limited to, ampicillin, tetracycline, kanamycin, hygromycin, bleomycin, chloramphenicol, streptomycin or phleomycin or a herbicide, to which the selectable marker of the cells provides resistance.

The fermentation may be performed as a batch, a repeated batch, a fed-batch, a repeated fedbatch or a continuous fermentation process. In a fed-batch process, either none or part of the compounds comprising one or more of the structural and/or catalytic elements, like carbon or nitrogen source, is added to the medium before the start of the fermentation and either all or the remaining part, respectively, of the compounds comprising one or more of the structural and/or catalytic elements are fed during the fermentation process. The compounds which are selected for feeding can be fed together or separate from each other to the fermentation process. In a repeated fed-batch or a continuous fermentation process, the complete start medium is additionally fed during fermentation. The start medium can be fed together with or separate from the feed(s). In a repeated fed-batch process, part of the fermentation broth comprising the biomass is removed at regular time intervals, whereas in a continuous process, the removal of part of the fermentation broth occurs continuously. The fermentation process is thereby replenished with a portion of fresh medium corresponding to the amount of withdrawn fermentation broth. Many cell cultures incorporate a carbon source, like glucose, as a substrate feed in the cell culture during fermentation. Thus, in one embodiment, the method of cultivating the microorganism comprises a feed comprising a carbon source. The carbon source containing feed can comprise a defined or a complex carbon source as described in detail herein, or a mixture thereof. The fermentation time, pH, conductivity, temperature, or other specific fermentation conditions may be applied according to standard conditions known in the art. In one embodiment, the fermentation conditions are adjusted to obtain maximum yields of the protein of interest.

In one embodiment, the temperature of the fermentation broth during fermentation is 30° C. to 45° C.

In one embodiment, the pH of the fermentation medium is adjusted to pH 6.5 to 9.

In one embodiment, the conductivity of the fermentation medium is after pH adjustment 0.1-100 mS/cm.

In one embodiment, the fermentation time is for 1-200 hours.

In one embodiment, fermentation is carried out with stirring and/or shaking the fermentation medium. In one embodiment, fermentation is carried out with stirring the fermentation medium with 50-2000 rpm.

In one embodiment, oxygen is added to the fermentation medium during cultivation, including, but not limited to, by stirring and/or agitation or by gassing, including but not limited to gassing with 0 to 3 bar air or oxygen. In one embodiment, fermentation is performed under saturation with oxygen.

In one embodiment, the fermentation medium and the method using the fermentation medium is for fermentation in industrial scale. In one embodiment, the fermentation medium of the present description may be useful for any fermentation having culture media of at least 20 litres, at least 50 litres, at least 300 litres, or at least 1000 litres.

In one embodiment, the fermentation method is for production of a protein of interest at relatively high yields, including, but not limited to, the protein of interest being expressed in an amount of at least 2 g protein (dry matter)/kg untreated fermentation medium, at least 3 g protein (dry matter)/kg untreated fermentation medium, of at least 5 g protein (dry matter)/kg untreated fermentation medium, at least 10 g protein (dry matter)/kg untreated fermentation medium, or at least 20 g protein (dry matter)/kg untreated fermentation medium.

In a preferred embodiment, the space-time-yield, carbon substrate flexibility and/or carbonconversion-efficiency of the production of one or more fine chemicals, preferably one or more oligosaccharides, is increased by at least 20%, 30%, 40%, 50%, 60%, 65% or 70% compared to the controls, i.e. the space-time-yield, carbon substrate flexibility and/or carbon-conversion-efficiency of a host cell that has cAMP levels that are not significantly changed and has an adenylate cyclase subject to regulatory activity and/or has unaltered abundance and/or activity of the Crr protein (SEQ ID NO: 26), of variants thereof or of endogenous protein(s) corresponding to the Crr protein.

Preferably, increased cAMP levels are to be understood to be increased by at least 5%, preferably at least 10%, 15%, 20%, 25%, 30%, 40%, 50%, 60%, 70%, 80%, 90% or more compared to the levels in unmodified host cell, for example those that have only adenylate cyclases under normal regulation and none of the de-regulated ones, and/or that have the normal crr gene locus or normal locus of the endogenous gene corresponding to the crr gene of E. coli and a corresponding protein at wildtype level of abundance or activity. For example, a modified microorganism modified to have reduced CRR protein levels will be compared in its cAMP level with the cAMP level of the unmodified microorganism. In another preferred embodiment the cAMP level of the host organism capable of producing one or more fine chemicals, preferably one or more oligosaccharides, is increased by a factor of 1.1, 1.2, 1.25, 1.3, 1.4, 1.5, 1.75, 2, 3, 4, 5, 6, 7, 8, 9, or 10 compared to normal level of the host organism.

The cAMP level of the host organism is preferably to be understood as the intracellular cAMP level, and more preferably the cytoplasmic cAMP level of a host organism. The cAMP level can be determined as disclosed herein above.

A further preferred embodiment is the use of a de-regulated adenylate cyclase and/or of the inactivation and/or the reduction in abundance of the Crr protein (SEQ ID NO: 26), of variants thereof or of the endogenous protein(s) corresponding to the Crr protein of SEQ ID NO: 26 for increasing space-time-yield, carbon substrate flexibility and/or carbon-conversion-efficiency of the production of one or more fine chemical by a host organism according to the invention.

A further embodiment is directed to the methods of the invention or the host cells of the invention wherein the activity and/or the abundance of the Crr protein (SEQ ID NO: 26), of variants thereof or of the endogenous protein(s) corresponding to the Crr protein of SEQ ID NO: 26 is reduced by 15% or 20%, more preferably 25%, 30%, 35% 40%, 50%, 60%, 70%, 80%, 85%, 90%, 92%, 94%, 95% or 98% or more in comparison to the controls i.e. those cells with a wildtype level of activity and/or abundance of the Crr protein (SEQ ID NO: 26), of variants thereof or of the endogenous protein(s) corresponding to the Crr protein of SEQ ID NO: 26.

### DESCRIPTION OF FIGURES

FIG. 1 shows a graphical display of the different lengths of the various DNA protein sequences useful in the methods and host cells of the inventions.

FIG. 2,

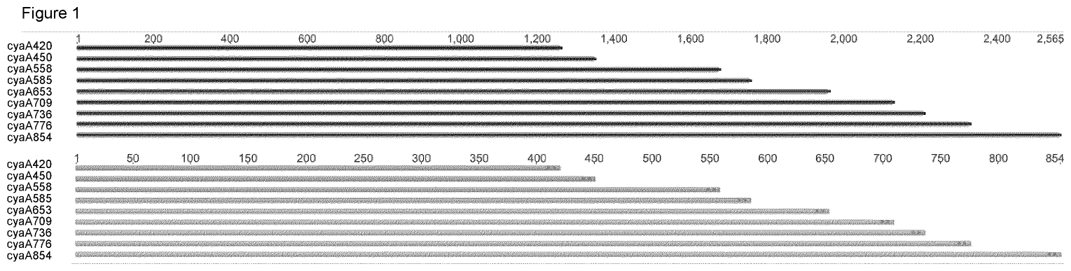

Part 1) is showing the alignment of the DNA sequences of SEQ ID NO: 1 to 8 and 10, showing the length of the different shortened cyaA DNA sequences compared to the longest variant of the full-length gene

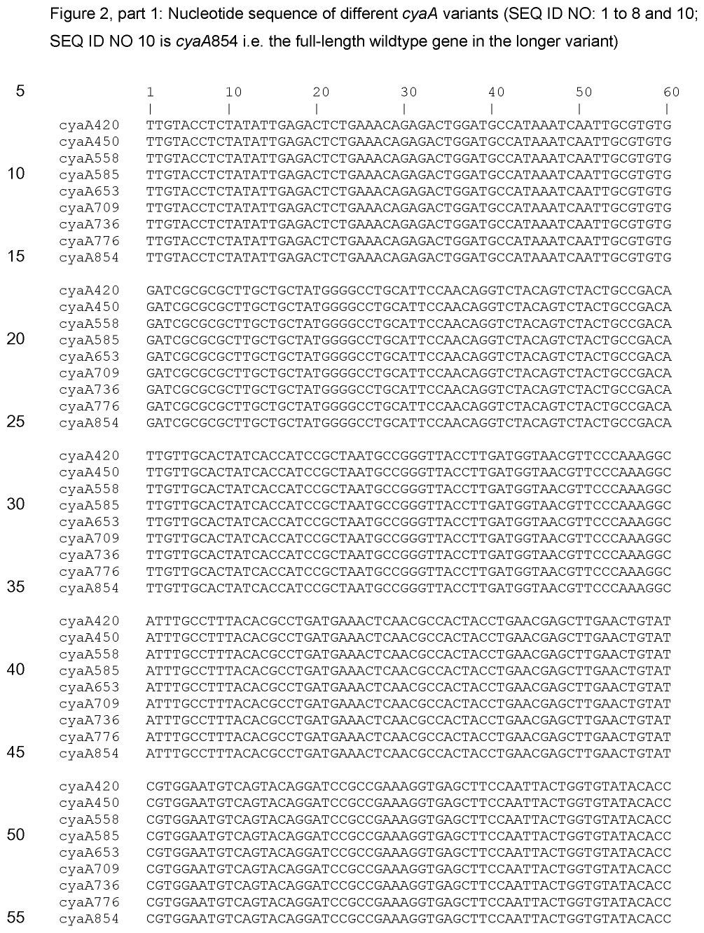

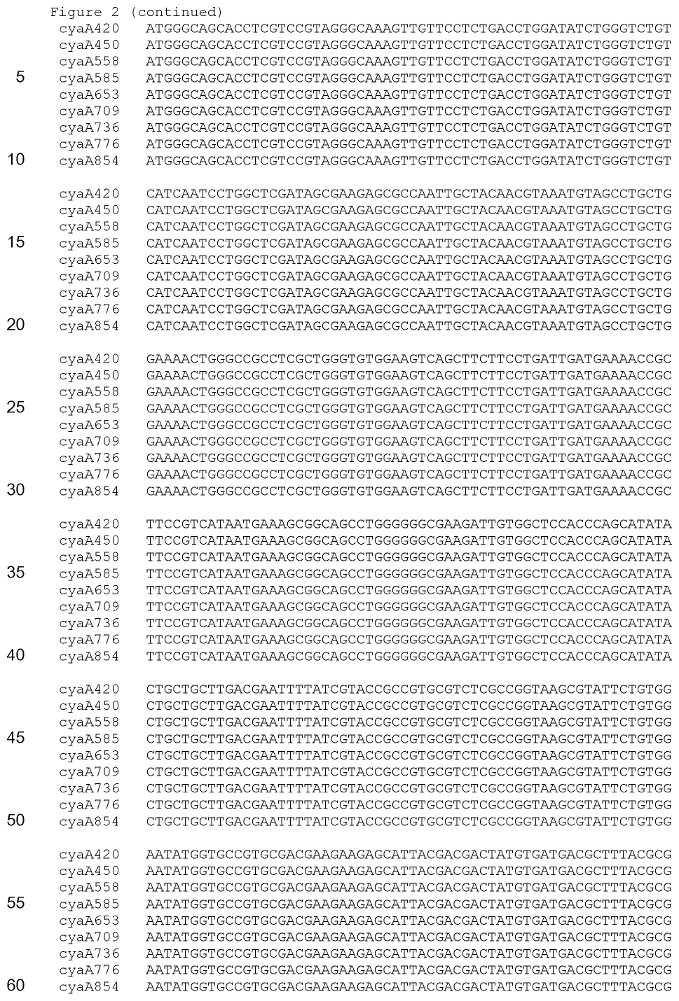

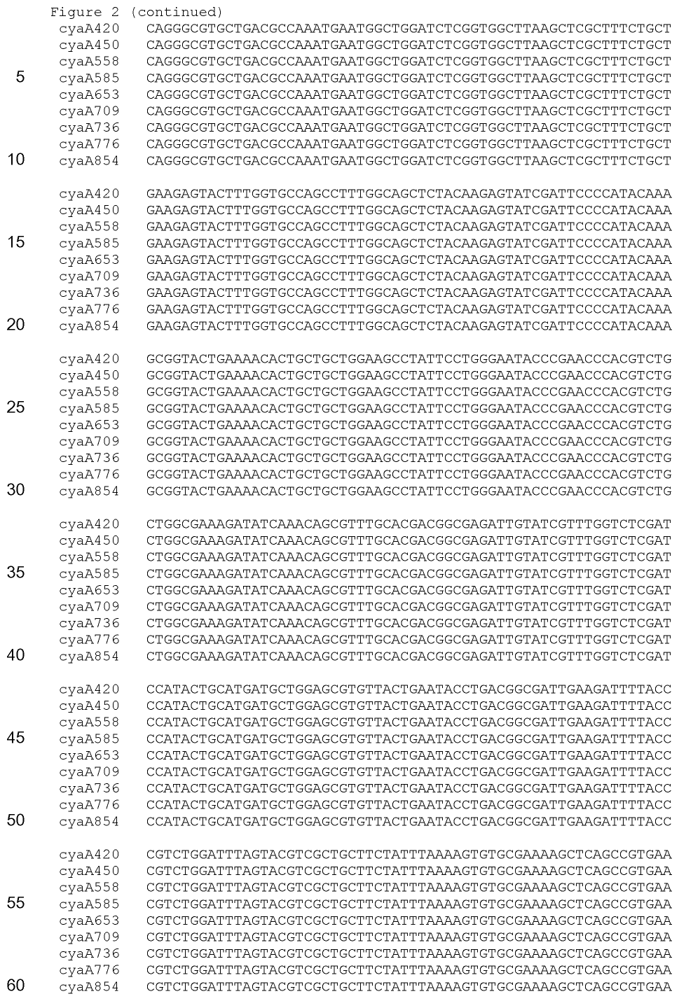

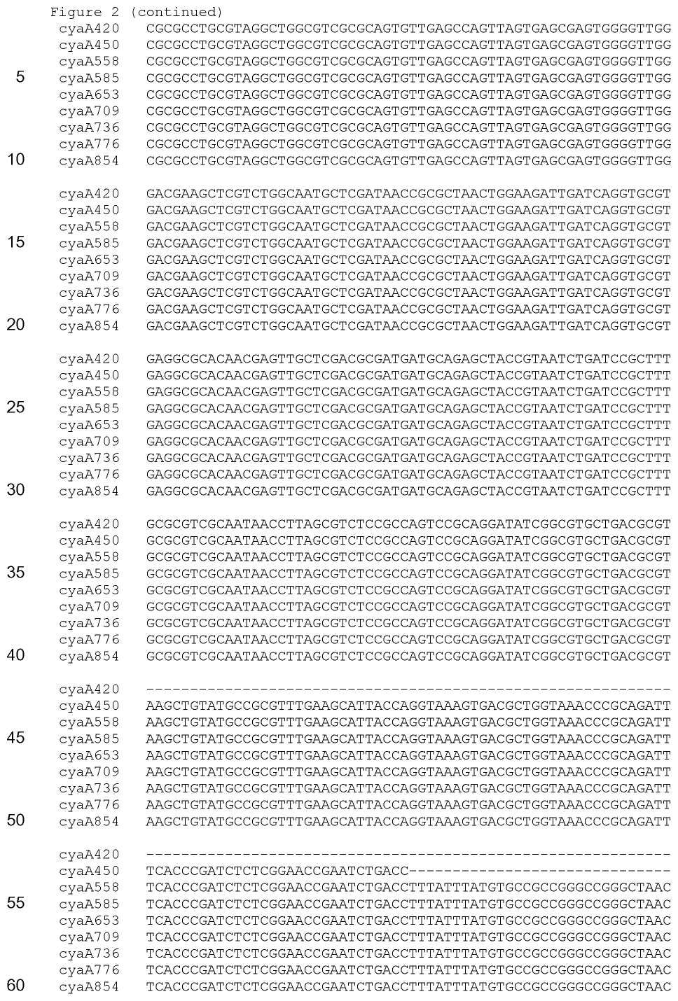

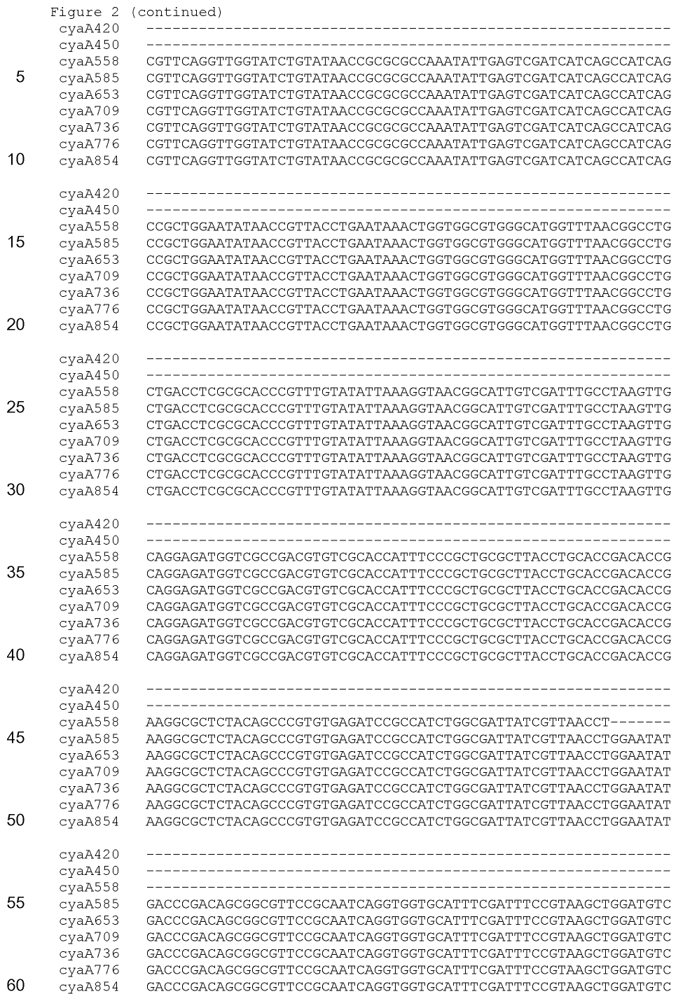

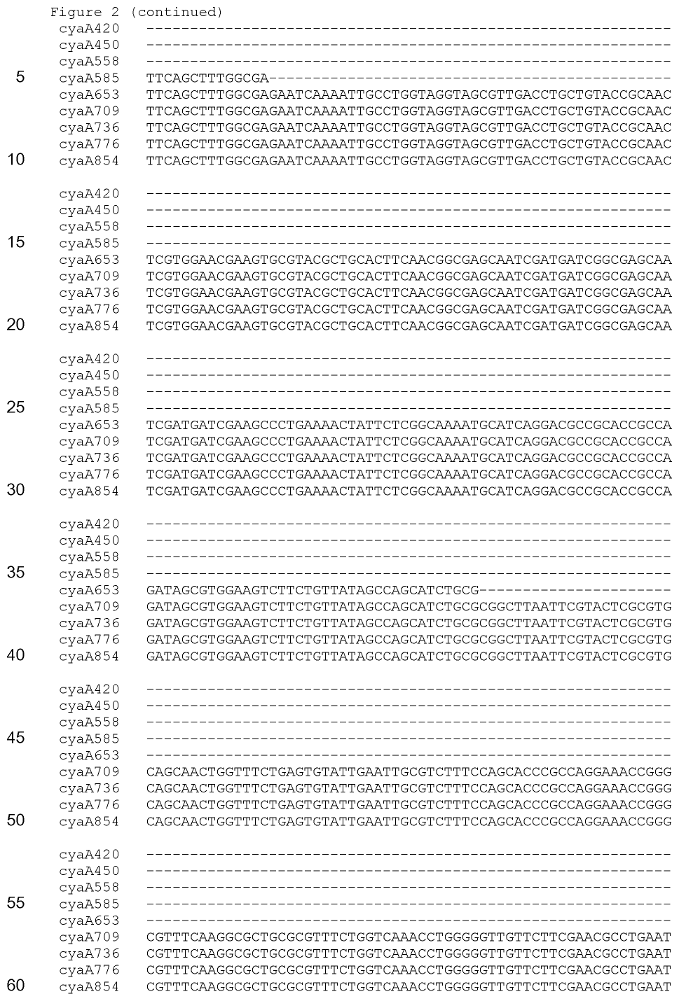

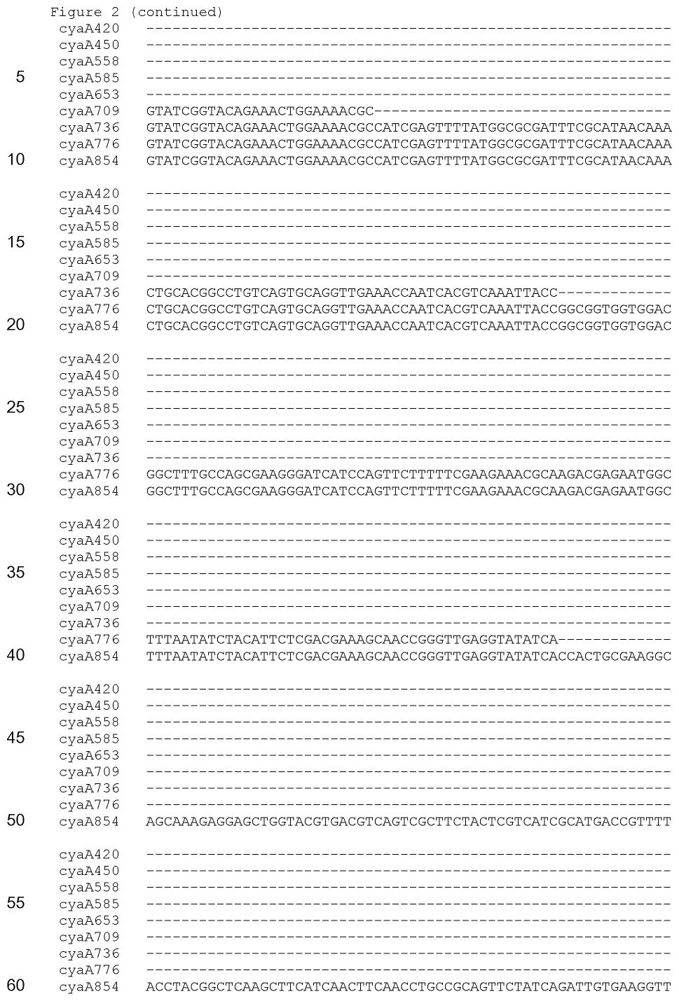

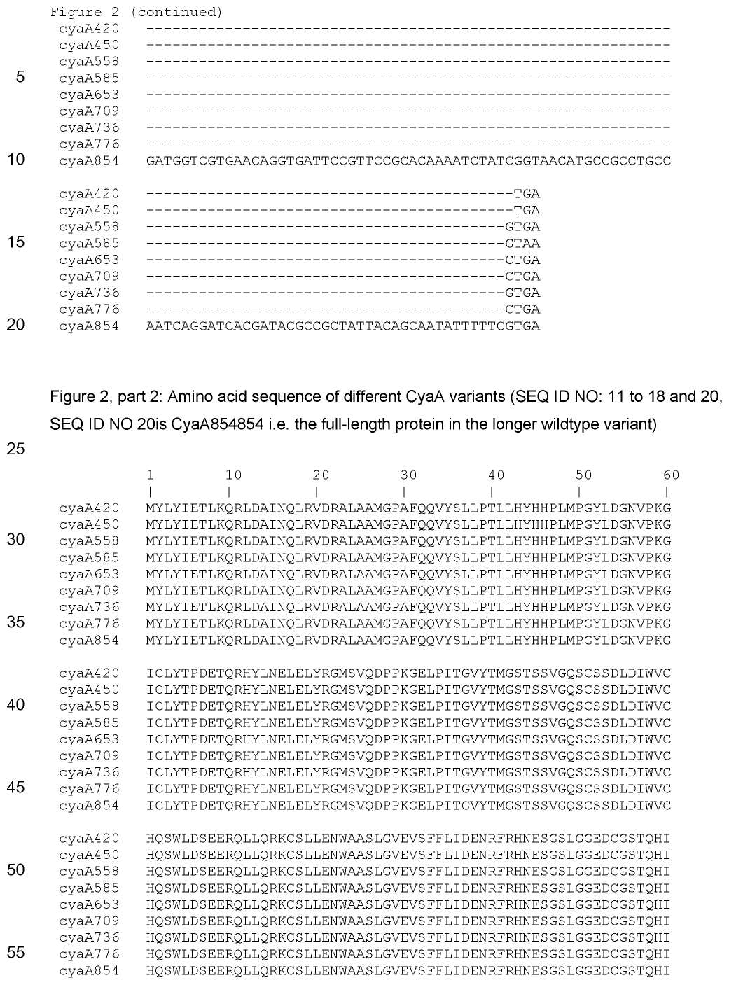

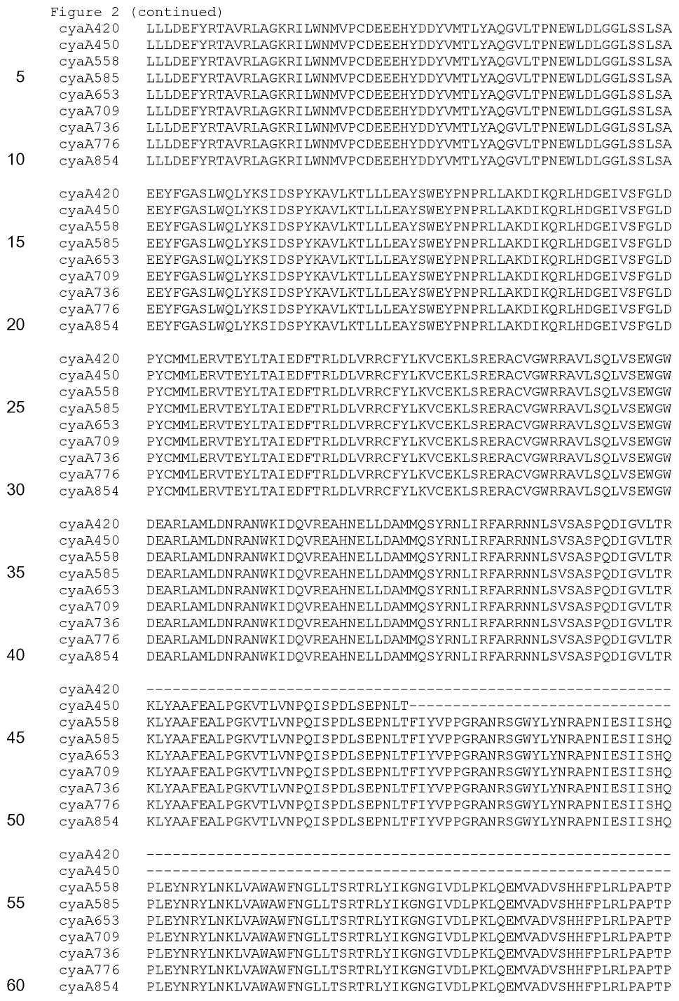

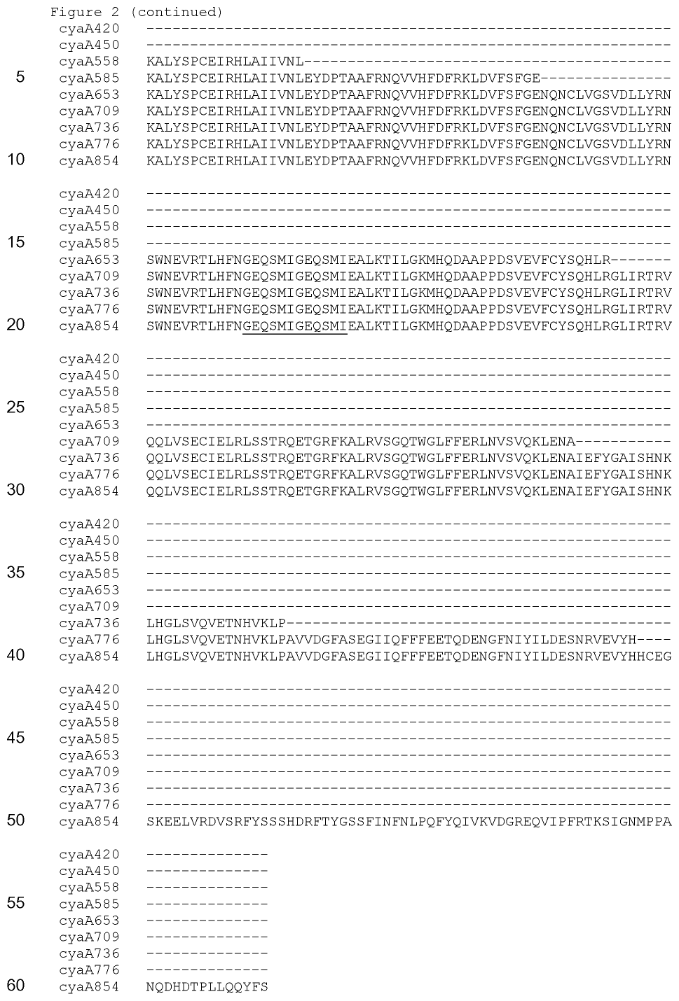

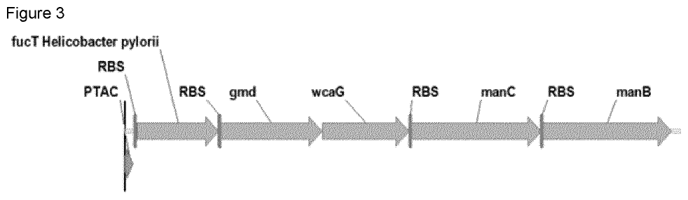

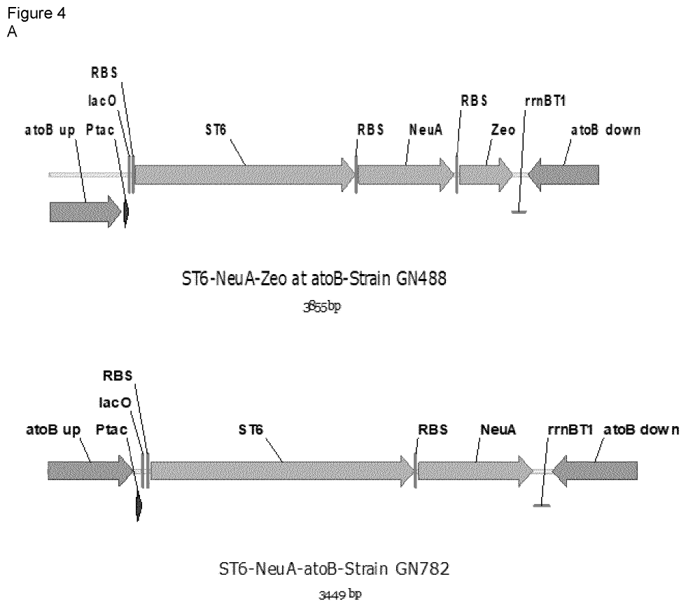

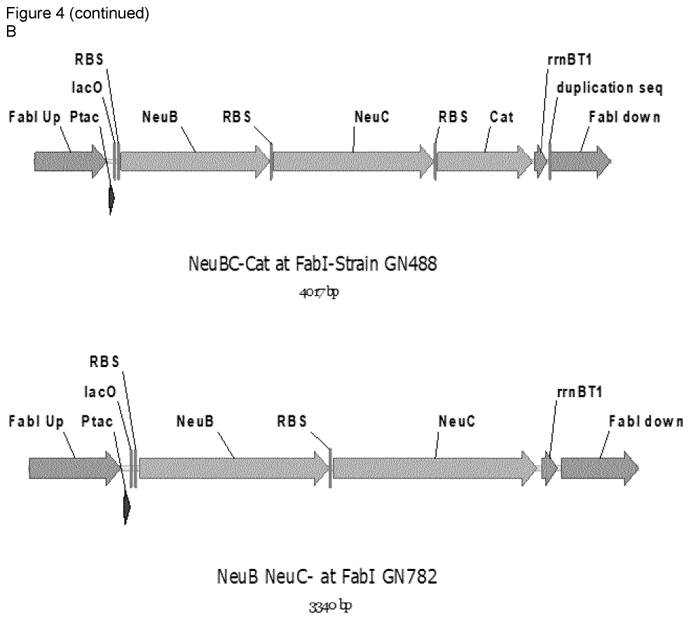

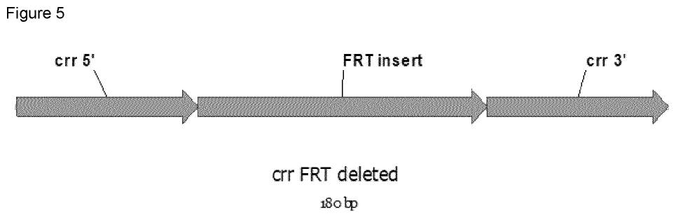

Part 2) is showing the alignment of the protein sequences of SEQ ID NO: 11 to 18 and 20, showing the length of the different shortened CyaA protein sequences compared to the longest variant of the full-length protein. In comparison the slightly shorter full-length wildtype protein of SEQ ID NO: 19 has only one GEQSMI motif instead of the duplicate GEQSMIGEQSMI (underlined in FIG. 2 part 2) of the 854-variant of the full-length adenylate cyclase.

FIG. 3 depicts an exemplary construct to create a 2′FL producing E. coli strain

FIG. 4

A depicts the first construct introduced to create a 6′-SL producing E. coli strain. The top picture is the construct in the strain without altered CyaA, the bottom is the one in the strain with de-regulated CyaA;

B: depicts the second construct used to create a 6′-SL producing E. coli strain. The top picture is the construct in the strain without altered CyaA, the bottom is the one in the strain with de-regulated CyaA.

FIG. 5 depicts the crr locus after deletion of the bulk of the crr gene as explained in the examples below in detail.

### FURTHER EMBODIMENTS

I. Method for the increase of space-time-yield of one or more fine chemicals in a host organism, the carbon-conversion-efficiency of the production of one or more fine chemicals by a host organism and/or carbon substrate flexibility of the production of one or more fine chemicals by a host organism by providing a de-regulated adenylate cyclase protein and/or inactivation and/or reduction in abundance of the Crr protein (SEQ ID NO: 26), of variants thereof or of the endogenous protein(s) corresponding to the Crr protein of SEQ ID NO: 26 in the host organism, wherein the space-time-yield, carbon-conversion-efficiency and/or carbon substrate flexibility are increased in the modified host organism compared to the non-modified host organism.
II. Method to increase the carbon substrate flexibility of the production of one or more fine chemicals by a host organism, wherein the cAMP levels in the host organism is increased compared to the non-modified host organisms.
III. Method to increase the carbon-conversion-efficiency of the production of one or more fine chemicals by a host organism, wherein the cAMP levels in the host organism is increased compared to the non-modified host organisms.
1. Method for the increase of space-time-yield of one or more fine chemicals produced by a host organism suitable for the production of one or more fine chemicals including the steps of increasing the Adenosine 3′,5′-cyclic monophosphate (cAMP, CAS Number: 60-92-4) levels of the host organism compared to the non-modified host organisms, maintaining the host organism in a setting allowing it to grow, growing the host organisms in the presence of substrates and under conditions suitable for the production of one or more fine chemicals and optionally separating one or more fine chemicals from the host organism or remainder thereof.
2. Method to increase the carbon substrate flexibility of the production of one or more fine chemicals by a host organism suitable for the production of one or more fine chemicals, including the steps of increasing the cAMP levels in the host organism compared to the non-modified host organisms, maintaining the host organism in a setting allowing it to grow, growing the host organisms in the presence of substrates and under conditions suitable for the production of one or more fine chemicals and optionally separating one or more fine chemicals from the host organism or remainder thereof.
3. Method to increase the carbon-conversion-efficiency of the production of one or more fine chemicals by a host organism suitable for the production of one or more fine chemicals, including the steps of increasing the cAMP levels in the host organism compared to the non-modified host organisms, maintaining the host organism in a setting allowing it to grow, growing the host organisms in the presence of substrates and under conditions suitable for the production of one or more fine chemicals and optionally separating one or more fine chemicals from the host organism or remainder thereof.
4. Method according to any of the preceding embodiments, wherein the cAMP level of the host organism is increased by
   a. Inactivating the regulatory activity found in a wildtype adenylate cyclase, and/or
   b. generating a mutated adenylate cyclase lacking the regulatory activity found in a wildtype adenylate cyclase, and/or
   c. introduction into the host organism of a mutated adenylate cyclase lacking the regulatory activity found in a wildtype adenylate cyclase; and/or
   d. reduction of the activity of the enzyme with the activity of a 3′,5′ cAMP phosphodiesterase (EC 3.1.4.53); and/or
   e. use of adenylate cyclase toxin of Bordetella pertussis or the adenylate cyclase domain of it, or a variant thereof; and/or
   f. inactivation and/or reduction in abundance of the Crr protein (SEQ ID NO: 26), of variants thereof or of the endogenous protein(s) corresponding to the Crr protein of SEQ ID NO: 26.
5. Method according to any of the preceding embodiments wherein the cAMP level of the host organism is increased in an inducible manner and the increase is compared to the host organisms without induction.
6. Method according to any of the preceding embodiments, wherein the mutated adenylate cyclase is introduced by introduction of a transgene.
7. Method according to any of the preceding embodiments, wherein the mutated adenylate cyclase or the adenylate cyclase with inactivated regulatory activity has a deletion compared to the wildtype form of the adenylate cyclase of the host organisms.
8. Method according embodiment 7, wherein the deletion is removing the regulatory part of the adenylate cyclase without disrupting the part producing cAMP.
9. Method according to embodiment 7 or 8, wherein the deletion is a deletion of the regulatory part of the protein that corresponds to C-terminal part of the adenylate cyclase encoded by an Escherichia coli cyaA gene, preferably that corresponds to C-terminal part of the cyaA pro-tein as provided in SEQ ID NOS:19 or 20, or an adenylate cyclase protein of at least 80% sequence identity to positions 1 to 412 preferably to positions 1 to 420 of the protein sequence provided as SEQ ID NO 19; and preferably the deletion is a deletion of the regulatory part of the protein that that corresponds to the part of the Escherichia coli adenylate cyclase that is subsequent to position 420, 450, 558, 582, 585, 653, 709, 736 or 776 of the protein sequence supplied in SEQ ID Nos: 19 or 20 more preferably subsequent to position 558, 582, 585, 653, 709, 736 or 776 of the protein sequence supplied in SEQ ID Nos: 19 or 20, and most preferably a deletion of the amino acids that correspond to the amino acids at the position 777 and following of SEQ ID NO 19 or 20.
10. The method according to any of the preceding embodiments, wherein the method includes the step of supplying the host organism with a carbon source, wherein the carbon source is a complex or a defined carbon source or combinations thereof.
11. Modified host cell suitable for the production of a fine chemical wherein the host cell is able to grow on glycerol and/or glucose and/or maltose and/or fructose and/or sucrose, preferably sucrose, glycerol, glucose and/or fructose, wherein the modified host cell has an adenylate cyclase with inactivated or absent regulatory activity, that has adenylate cyclase activity, and/or inactivation and/or reduction in abundance of the Crr protein (SEQ ID NO: 26), of variants thereof or of the endogenous protein(s) corresponding to the Crr protein of SEQ ID NO: 26, and wherein the host organism has increased cAMP level compared to a non-modified host cell, wherein the non-modified host cell is unable to grow substantially on glycerol and/or glucose and/or maltose and/or fructose and/or sucrose.
12. Modified host cell of embodiment 11, wherein at least one adenylate cyclase protein corresponding to the protein encoded by the cyaA gene of Escherichia coli is lacking a regulatory activity, preferably lacking the part that corresponds to C-terminal part of the cyaA protein as provided in SEQ ID NOS:19 or 20, or an adenylate cyclase protein of at least 80% sequence identity to positions 1 to 412 more preferably an adenylate cyclase protein of at least 80% sequence identity to positions 1 to 420, of the protein sequence provided as SEQ ID NO 19 or 20, and preferably lacking the part of the adenylate cyclase that corresponds to the Escherichia coli adenylate cyclase part that is subsequent to position 420, 450, 558, 585, 653, 709, 736 or 776, more preferably 450, 558, 585, 653, 709 or 736 of the protein sequence supplied in SEQ ID Nos: 19 or 20 even more preferably subsequent to position 558, 582, 585, 653, 709, 736 or 776 of the protein sequence supplied in SEQ ID Nos: 19 or 20, and most preferably a deletion of the amino acids that correspond to the amino acids at the position 777 and following of SEQ ID NO 19 or 20.
13. Any of the preceding embodiments, wherein the host cell is a bacterial of fungal host cell, preferably a bacterial cell, more preferably a bacterial cell, even more preferably a gram-negative bacterial cell, most preferably an Escherichia coli cell
14. Use of de-regulated adenylate cyclase and/or inactivation and/or reduction in abundance of the Crr protein (SEQ ID NO: 26), of variants thereof or of the endogenous protein(s) corresponding to the Crr protein of SEQ ID NO: 26 for increasing space-time-yield, carbon substrate flexibility and/or carbon-conversion-efficiency of the production of one or more fine chemical by a host organism.
15. Any of the preceding embodiments wherein at least one fine chemical is a human milk oligosaccharide, preferably a neutral or sialylated HMO, more preferably 2′-fucosyllactose (2′-FL), 3′-fucosyllactose (3′-FL), lacto-N-tetraose (LNT), lacto-N-neotetraose (LNnT), difucosyllactose (2,3-DFL) or 3′-sialyllactose (3′-SL), 6′-sialyllactose (6′-SL) or the method of any of the preceding embodiments, wherein the method includes supplying the host organism with a carbon source, wherein the carbon source is one or more of the following: a complex or a defined carbon source, preferably glucose, fructose, galactose, xylose, arabinose, sucrose, maltose, dextrin, lactose, gluconate, more preferably glycerol, glucose or mannose, and even more preferably glucose or glycerol.
16. A method for the production of an oligosaccharide by conversion of a source of carbon in a fermentative process comprising the following steps:
   Culturing a microorganism genetically modified for the production of oligosaccharides in an appropriate culture medium comprising at least one source of carbon
   Recovering the human milk oligosaccharide from the culture medium, wherein said genetically modified microorganism comprises functional genes coding for a PTS carbohydrate utilization system and wherein in said genetically modified microorganism the abundance of the Crr protein (SEQ ID NO: 26), of variants thereof or of the endogenous protein corresponding to the Crr protein of SEQ ID NO: 26 is decreased and/or a deregulated adenylate cyclase as defined in any of the previous embodiments is present in the microorganism.
17. Any of the preceding embodiments, wherein the source of carbon is selected among the group consisting of glycerol, monosaccharides and disaccharides
18. Any of the preceding embodiments wherein the levels of Adenosine 3′,5′-cyclic mono-phosphate (cAMP, CAS Number: 60-92-4) are increased compared to a microorganism without alteration of the Crr protein (SEQ ID NO: 26), of variants thereof or of the endogenous protein corresponding to the Crr protein of SEQ ID NO: 26.
19. Genetically modified microorganism for an enhanced production of fine chemicals wherein said genetically modified microorganism is capable to produce human milk oligosaccharides wherein said genetically modified microorganism comprises functional genes coding for a PTS carbohydrate utilization system and wherein in said genetically modified microorganism the expression of the Crr protein is decreased, preferably at least substantially decreased.
20. A microorganism according to embodiment 19 wherein the gene encoding the Crr protein is attenuated or deleted in said genetically modified microorganism.
21. A microorganism according to any of the preceding embodiments, wherein the microorganism is selected among the group consisting of Enterobacteriaceae.

### EXAMPLES

In the examples given below, methods well known in the art were used to construct E. coli strains containing replicating vectors and/or various chromosomal deletions, and substitutions using homologous recombination well described by Datsenko & Wanner, (2000) for Escherichia coli. In the same manner, the use of plasmids or vectors to express or over-express one or several genes in a recombinant microorganism are well known by the man skilled in the art.

Methods

Introduction of a DNA construct or vector into a host cell can be performed using techniques such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. General transformation techniques are known in the art (see, e.g., Current Protocols in Molecular Biology (F. M. Ausubel et al. (eds) Chapter 9, 1987; Sambrook et al, Molecular Cloning: A Laboratory Manual, 2nd ed., Cold Spring Harbor, 1989; and Campbell et al, Curr. Genet. 16:53-56, 1989, which are each hereby incorporated by reference in their entireties, particularly with respect to transformation methods). The expression of heterologous polypeptide in Trichoderma is described in U.S. Pat. Nos. 6,022,725; 6,268,328; 7,262,041; WO 2005/001036; Harkki et al., Enzyme Microb. Technol. 13:227-233, 1991; Harkki et al, Bio Technol 7:596-603, 1989; EP 244,234; EP 215,594; and Nevalainen et al, “The Molecular Biology of Trichoderma and its Application to the Expression of Both Homologous and Heterologous Genes,” in Molecular Industri-al Mycology, Eds. Leong and Berka, Marcel Dekker Inc., NY pp. 129-148, 1992, which are each hereby incorporated by reference in their entireties, particularly with respect to transformation and expression methods). Reference is also made to Cao et al, (Sd. 9:991-1001, 2000; EP 238023; and Yelton et al, Proceedings. Natl. Acad. Sci. USA 81:1470-1474, 1984 (which are each hereby incorporated by reference in their entireties, particularly with respect to transformation methods) for transformation of Aspergillus strains. The introduced nucleic acids may be integrated into chromosomal DNA or maintained as extrachromosomal replicating sequences.

Examples with increased cAMP and deregulated adenylate cyclase activity
1. Creation of shortened cyaA DNA constructs
   Shortened DNA cyaA constructs were prepared by generating synthetic DNA constructs with homology for integration and introducing TAA stop codons into the coding sequence of the cyaA gene by gene synthesis. These genetic constructs were then introduced into the genome of the E. coli strain by homologous recombination as described Wang J, et al. 2006, Mol. Biotechnol., 32, 43
2. Strain construction
   Genetically modified microorganisms with enhanced production of oligosaccharides (e.g. HMOs) are disclosed in patent applications published as WO 2016/008602, WO2013/182206, EP2379708, U.S. Pat. No. 9,944,965, WO2012/112777, WO2001/04341 and US2005019874. All of these disclosures are herein incorporated by reference.

### 2′-FL Producing Microorganism

An E coli strain 2′-FL overproducing strain was constructed as follows: In the well characterized E. coli strain JM109, an artificial operon was constructed containing the following genetic elements: a PTAC promoter, an artificial ribosomal binding site (RBS), the fucT2 gene (derived from Helicobacter pylori strain 26695, Wang et al, Mol. microbiol. 1999, 31 1265-1274)), an artificial ribosomal binding site, the gmd gene (de-rived from E. coli K12), the wcaG gene with its authentic ribosomal binding site (derived from E. coli K12), an artificial ribosomal binding site (RBS), the manC gene (derived from E. coli K12) with an adapted codon usage), an artificial ribosomal binding site (RBS), the manB gene (derived from E. coli K12, with an adapted codon usage) and a transcriptional terminator rrnBT1 derived from the 16s rRNA locus of E. coli, using the well-known lambda red technology (e.g. described by Datsenko I and Wanner B. PNAS, 2000 97 (12) 6640-6645, Wang J, et al. 2006, Mol. Biotechnol., 32, 43). The artificial operon was integrated in into the fuc locus of E. coli in which the genes including fuc I and K were deleted. An exemplary construct for creating a 2′FL producing strain is shown as SEQ ID NO: 21.

The truncated adenylate cyclase gene sequences of SEQ ID NO: 1 to 8 were introduced via homologous recombination using the lambda-red technology into the Escherichia coli host cells. An exemplary construct for creating a 2′FL producing strain is shown as SEQ ID NO: 21.

### 6′-SL Producing Microorganism

An E coli strain strain overproducing 6′-SL was constructed as follows: In the well characterized E coli strain W3110, the genes lacZ gene coding for the beta galactosidase LacZ and the lacA gene coding for the acetyltransferase LacA, the genes coding for the nan genes nanAETK were deleted in that all coding sequence was deleted suing the well-known lambda red technology (e.g. described by Datsenko I and Wanner B. PNAS, 2000 97 (12) 6640-6645, Wang J, et al. 2006, Mol. Biotechnol., 32, 43), while the lacI allele was replaced by the known laclq allele. An artificial operon (see SEQ ID NO: 22) was integrated immediately adjacent to the atoB gene of the strain W3110. The artificial operon contained the following genetic elements, a PTAC promoter, an artificial ribosomal binding site (RBS), the St6 gene (derived from Photobacterium spp. ISH 224), an artificial ribosomal binding site, the neuA gene (derived from Campylobacter jejuni ATCC 43438), an artificial ribosomal binding site (RBS), the zeocin resistance genes and a transcriptional terminator rrnBT1 derived from the 16s rRNA locus of E. coli. In addition, an artificial operon was integrated immediately adjacent to the fabl gene. The artificial operon contained the PTAC promoter, an artificial ribosomal binding site (RBS), the neuB gene (derived from Campylobacter jejuni ATCC 43438, see SEQ ID NO: 23), an artificial ribosomal binding site, the neuC gene (derived from Campylobacter jejuni ATCC 43438, see SEQ ID NO: 24), an artificial ribosomal binding site (RBS), the chloramphenicol resistance cassette (CAT) and a transcriptional terminator rrnB derived from the 16s rRNA locus of E. coli.
This 6′-SL producing strain will be called GN488.
Another E coli strain with the designation GN782 was constructed based on the Strain GN488. The resistance genes zeocin and CAT were deleted from the artificial operon of genome of the strain GN488 again using the lambda red technology. In addition, the cyaA was changed in that a stop codon was introduced at codon 582 resulting in a translated protein which has a length of 581 amino acids.
3. De-regulated adenylate cyclase: Space-time yield in the production of HMO
   Fermentation system and procedure
   Fermentation conditions:
   A fermentation medium was chosen based on the described examples of E. coli fermentation and can be found in: (Riesenberg et al. (1991), Journal of Biotechnology 20, 17-27, D. J. Korz, et al. 1995), J. Biotechnol., 39 pp. 59-65, Biener, R. et al. 2010, Journal of Biotechnology 146(1-2), pp. 45-53. Specifically the medium was altered for the production of oligosaccharides based on lactose in that lactose was added in different concentrations ranging from 20-100 g/l dependent on the experiment.
   For analysing strain performance in regard to carbon-conversion-efficiency as well as space-time-yield the following systems were used: AMBR® 250 system and 4 l Biostat® fermenters (both from Sartorius AG, Otto-Brenner-Str. 20, D-37079 Göttingen, Germany). Generally speaking, fermentations were typically conducted under the following regime: A seed culture was grown from a frozen stock. The seed culture was inoculated into the respective fermentation system (AMBR or Biostat) before its carbon content was fully utilized. Alternatively, the main culture was started directly from the frozen stock. The fermentation in the fermentation system was conducted in a fed batch mode, i. e. that a fermentation undergoes two stages—the initial one in which a batched amount of carbon source is being utilized, and the following one in which the carbon source is fed throughout the fermentation under conditions where no or only low amounts of carbon source will accumulate in the fermentation broth.
   The seed culture (minimal medium with 10 ml/L trace element solution and 65 g/L glycerol) is inoculated with 1 ml WCB culture (stored in a frozen state).
   The seed culture is transferred to the main culture in that an inoculation volume ratio between 1 and 10% are applied.
   The main fermentation medium consists of the following media composition: Minimal medium: citric acid 1.1 g/L, glycerol 10.8 g/L, KH2PO4 15.5 g/L, (NH4)2SO4 4.6 g/L, Na2SO4 3 g/L, MgSO4*7H2O 1.5 g/L, thiamine 0.02 g/L, Vitamin B12 0.0001 g/L, 0.5 mM IPTG. The Trace element solution consist of: Na2-EDTA*2H2O 4 g/L, CaSO4*2H2O 1 g/L, ZnSO4*7H2O 0.3 g/L, FeSO4*7H2O 3.7 g/L, MnSO4*H2O 0.2 g/L, CuSO4*5H2O 0.15 g/L, Na2MoO4*2H2O 0.04 g/L, Na2SeO4 0.04 g/L. The trace metal solution is applied at an amount of 30 ml/l of fermentation medium.
   After inoculation the fermentation is started and when the measured CTR is exceeding 40 mmol/Lh, the feeding of carbon source such as glycerol (86% w/w concentration) or glucose (60% w/w concentration) is initiated. Carbon source feed rates may vary between 2-8 g/I carbon source per litre of initial fermentation broth volume per hour. Care is taken that carbon source does not accumulate throughout the fermentation process. In the main fermentation stage, the dissolved oxygen concentration (pO2) is controlled at >20% by controlling agitation as well as gas addition. pH is maintained at values ranging from 6.1 to 6.9 and more specifically at pH 6.7 using the base NH4OH in a solution of 15% NH4OH aq. Results in both fermentation systems in regard to the parameters mentioned (carbon-conversion-efficiency and space-time-yield) were found to be fully superimposable and can be understood fully interchangeable.
   Surprisingly the cAMP overproduction cells with the truncated cyaA gene resulting in a functional, de-regulated CyaA protein did grow and produce 2′-Fucosyllactose (2′-FL) well on glycerol. In contrast to this, the a cyaA deletion mutant (from the Keio collection, Baba T, Ara T, Hasegawa M, Takai Y, Okumura Y, Baba M, Datsenko K A, Tomita M, Wanner B L, Mori H (2006) Construction of Escherichia coli K-12 in-frame, single-gene knockout mutants: the Keio collection. Mol. Syst. Biol. 2: 2006 0008) having no functional adenylate cyclase was found to be unable to grow on glycerol The unmodified E. coli cells with an adenylate cyclase with a regulatory part are growing more slowly than in the host cells with the de-regulated adenylate cyclase and hence increased cAMP production, and 2′-FL production is lower in the unmodified cells, carbon-conversion efficiency and space/time yield are also decreased in comparison to the host cells with the de-regulated adenylate cyclase and hence increased cAMP production.
   2′-FL

TABLE 2A
Carbon-conversion-efficiency in 2′-FL production.
FL is the abbreviation for full-length

| | | | Carbon-conversion- |
|---|---|---|---|
| | | | efficiency |
| | | | (CCE) g 2′-FL/g |
| | | | carbon source |
| | Protein ending with | 2′-FL (relative | (relative values |
| Protein | AA number | values in %) | in %) |
| FL cyaA | 854 | 100 | 100 |
| cyaA420 | 420 | 108 | 110 |
| cyaA450 | 450 | 121 | 114 |
| cyaA585 | 585 | 123 | 119 |
| cyaA558 | 558 | 119 | 123 |
| cyaA653 | 653 | 125 | 122 |
| cyaA709 | 709 | 128 | 116 |
| cyaA736 | 736 | 112 | 117 |
| cyaA776 | 776 | 126 | 124 |

Typically, when the BioStat® and the AMBR® vessels were used, the carbon source was added continuously or in repeated additions. In principle a typical amount of glucose or glycerol can be added once at the start of the main culture, which is advantageous when e.g. shaking flask are used for the fermentation.
The space-time-yield was increased when glucose or glycerol was used as a carbon source for the strains with the de-regulated cyaA gene and hence increased cAMP levels.

TABLE 2B
Space-time-yield in 2′-FL production

| | Space-time- | Space-time- |
|---|---|---|
| | yield on glucose | yield on glycerol |
| | relative values | relative values |
| | [%] to wildtype | [%] to wildtype |
| | (=100%) | (=100%) |
| cyaA854 (wt) | 100 | 100 |
| cyaA585 | 140 | 116 |

Similar results were achieved with the E. coli strain producing 6′-Sialyllactose instead of 2′-FL, for these strains see example 1 and 2 above.
4. Increased carbon source flexibility of 2′-FL producing strains
   Carbon sources are batched into the medium as well as fed during the feed phase ranging from 2 h- to 100 h. The carbon sources are applied either in a pure fashion (e.g. glycerol) or diluted in water (glycerol as well as other carbon sources). The feed rate of the carbon source is adapted to the stirring and aeration conditions of the fermenter.
   In the course of the fermentation, samples were taken and analyzed by isocratic HPLC elution method.
   Carbon source flexibility analysis for 2′-FL production was performed using the following media composition:
   20 mL of medium (10 g/L of the respective carbon source, 5 g/L lactose, 1 g/L (NH₄)₂H-citrate, 2 g/L Na₂SO₄, 2.68 g/L (NH₄)₂SO₄, 0.5 g/L NH₄Cl, 14.6 g/L K₂HPO₄, 4 g/L NaH₂PO₄*H₂O, 0.5 g/L MgSO₄*7H₂O, 10 g/mL MnSO₄, 3 mL trace metal solution consisting of 8.0 g/L Na₂-EDTA*2H₂O, 1 g/L CaSO₄*2H₂O, 0.3 g/L ZnSO₄*7H2O, 7.4 g/L (NH₄)₂Fe(SO₄)₂, 0.2 g/L MnSO₄*H₂O, 0.15 g/L CuSO₄*5H₂O, 0.04 g/L Na₂MoO₄*2H₂O, 0.04 g/L Na₂SeO₄, 10 mg/L thiamine*HCl, 0.1 mg/L vitamin B12, 1 mM IPTG, pH 7.0) in a 100 mL baffled shake flask were inoculated with an overnight culture of a 2′-FL producing strain as in example 2 (in the above described medium without lactose and IPTG) to a start OD of 0.5 and incubated for 24 hours in the above described medium including lactose and IPTG as given above at 200 rpm at 37° C. Samples were taken and analysed for carbon utilization and product formation.
   Carbon sources were chosen from the following list:
   Glucose, Glycerol, Mannose, Fructose,
      Table 3: Relative carbon conversion rates for different carbon sources

TABLE 3
Relative carbon conversion rates for different carbon sources

| | Carbon- | Carbon- | Carbon- | Carbon- |
|---|---|---|---|---|
| | conversion- | conversion- | conversion- | conversion- |
| | efficiency | efficiency | efficiency | efficiency |
| | (g Fucose/ | (g Fucose/ | (g Fucose/ | (g Fucose/ |
| | g fructose) | g mannose) | g glucose) | g glycerol) |
| | relative to | relative to | relative to | relative to |
| | wildtype | wildtype | wildtype | wildtype |
| | (=100%) | (=100%) | (=100%) | (=100%) |
| cyaA854 (wt) | 100 | 100 | 100 | 100 |
| cyaA585 | 76 | 112 | 133 | 147 |

5. 6′-sialyllactose (6′-SL) producing strains
   Strains GN488 and strain GN782 of example 2 were grown in a Biostat® vessel containing the medium as described in example 3.

TABLE 4
Increased carbon-conversion-efficiency and
space-time-yield in the production of 6′-SL

| | | | Carbon-conversion- | |
|---|---|---|---|---|
| | | | efficiency (CCE) g | |
| | | | 6SL/g carbon source | space-time- |
| | Relevant | Carbon | relative values | yield relative |
| strain | genotype | source | [%] | values [%] |
| GN488 | cyaA848 | glycerol | 100 | 100 |
| GN782 | cyaA 582 | glycerol | 138 | 133 |
| | stop | | | |

The results showed that the surprising effects on carbon-conversion-efficiency and spacetime-yield are transferable to other HMO producing strains and the broad applicability of the de-regulated adenylate cyclase to increase cAMP levels since yet another version of the de-regulated CyaA protein corresponding to the amino acids 1 to 581 of the full-length CyaA protein with 848 amino acids (SEQ ID NO: 19) was successfully used. Furthermore, when the strain holding the cyaA585 version of the protein (SEQ ID NO:14) was tested, the space-time-yield of 6′-SL was similarly increased over the strain with an unmodified CyaA protein.
6. cAMP feeding experiments
   An E. coli strain of the Keio collection (Baba T, Ara T, Hasegawa M, Takai Y, Okumura Y, Baba M, Datsenko K A, Tomita M, Wanner B L, Mori H (2006) Construction of Escherichia coli K-12 in-frame, single-gene knockout mutants: the Keio collection. Mol. Syst. Biol. 2: 2006 0008) with a deletion of the cyaA gene shows the normal poor growth on glycerol as carbon source. This strain is grown in the presence of glycerol and cAMP and the growth of the deletion strain is improved. The 2′-FL producing host cells with a shortened adenylate cyclase of examples 1 and 2 above shows increased 2′-FL production on medium containing glycerol compared to the cells with an unmodified cyaA gene only. If the latter are supplied with cAMP, the production of 2′-FL is increased.

Examples with Altered cAMP Signalling and PTS

### Example 7: Construction of a Strain Overproducing 2′-FL

An E coli strain overproducing 2′-FL with wildtype adenylate cyclase and wildtype crr gene was constructed as described in example 2 above.

Construction of an Overproducing Strain Carrying a Deletion in the Crr Gene

An E. coli strain 2′-FL overproducing strain carrying a deletion in the crr gene was constructed as follows: The well-known method described by Datsenko I and Wanner B. PNAS, 2000 97 (12) 6640-6645, Wang J, et al. 2006, Mol. Biotechnol., 32, 43A was used to replace the intact full length crr gene in the 2′FL producing strain with a genetic construct consisting of 50 bp of the 5′ coding region of the crr beginning with the transcriptional start site, a resulting FRT site from the FLP recombination event, and 50 bp of the crr gene ending with the TAA sequence of the translational stop codon. The resulting gene (SEQ ID NO: 29) therefore is not coding for an active crr protein since it is lacking 410 bp of its coding region.

The deletion of the crr gene was confirmed using the primers given in SEQ ID NO 3 & 4.

### Example 8: Construction of a Strain Producing 6′SL Having a Deletion in the Crr Gene

The strain GN488 overproducing 6′-SL was created as described in example 2 above and used for further modifications. In this strain, the deletion of the crr gene (SEQ ID NO:1) in Escherichia coli strains was made by P1 viral transduction followed by selection on kanamycin containing agar plates.

A P1 lysate was made of the delta crr strain (JW2410/b2417) crr::kan) from the Keio collection (Baba et al. 2006, Mol Syst Biol. 2:2006.0008). The crr:Kan P1 lysate was used to transduce the strains described in examples 1 and 2 and the transductants were selected on agar plates containing kanamycin. Colonies were screened by PCR using primers selective for the upstream and downstream region of crr to confirm the deletion of crr. A colony with the expected bandsize indicating the correct deletion of the crr gene.

The deletion of the crr gene (SEQ ID NO:1) in Escherichia coli strains was made by P1 viral transduction (Miller, J. H. 1992. A Short Course in Bacterial Genetics: A Laboratory Manual and Handbook for Escherichia coli and Related Bacteria. Cold Spring Harbor Laboratory Press, Cold Spring Harbor, N.Y.) followed by selection on kanamycin-citrate containing agar plates.

A P1 lysate was made of strain (JW2410/b2417) (delta crr::kan(FRT)) from the Keio collection (Baba et al. 2006, Mol. Syst. Biol. 2:2006.0008). The delta crr:Kan P1 lysate was used to transduce the strains described in example 1 & 2 (2′-FL and 6′-SL strains, respectively) and the transductants were selected on agar plates containing kanamycin-citrate. Colonies were screened by PCR using primers Crr ver.F (SEQ ID NO: 27) and Crr ver.R (SEQ ID NO: 28) to confirm the deletion of crr. One correct colony was selected and designated as Ec 6′-SL delta crr.

### Example 9: Increased Space-Time Yield in the Production of HMO

Fermentation conditions, system and procedures were as described above under example 3 above.

TABLE 5
Space/time yield of 2′-FL production with wildtype
(wt) crr or crr functional gene deletion (delta crr)

| | | Carbon | space-time- |
|---|---|---|---|
| | Relevant | source | yield (relative |
| strain | genotype | applied | values [%]) |
| N8_2 | Crr wt | Glucose | 100 |
| N16_1 | Delta crr | Glucose | 146 |
| N8_2 | crr wt | Glycerol | 100 |
| N16_1 | Delta crr | Glycerol | 227 |

Typically, when the BioStat® and the AMBR® vessels were used, the carbon source was added continuously or in repeated additions. In principle a typical amount of glucose or glycerol can be added once at the start of the main culture, which is advantageous when e.g. shaking flask are used for the fermentation.

### Example 10: Increased Carbon Source Flexibility of Modified Strains Producing 2′FL

Carbon sources are batched into the medium as well as fed during the feed phase ranging from 2 h- to 100 h. The carbon sources are applied either in a pure fashion (e.g. glycerol) or diluted in water (glycerol as well as other carbon sources). The feed rate of the carbon source is adapted to the stirring and aeration conditions of the fermenter.

In the course of the fermentation, samples were taken and analysed by isocratic HPLC elution method.

Carbon source flexibility analysis was performed using the following media composition:

Carbon sources were chosen from the following list:

Glucose, glycerol, mannose, fructose

20 mL of medium (10 g/L of the respective carbon source, 5 g/L lactose, 1 g/L (NH₄)₂H-citrate; 2 g/L Na₂SO₄, 2.68 g/L (NH₄)₂SO₄, 0.5 g/L NH₄Cl, 14.6 g/L K₂HPO₄, 4 g/L NaH₂PO₄*H₂O, 0.5 g/L MgSO₄*7H₂O, 10 g/mL MnSO₄, 3 mL trace metal solution consisting of 8.0 g/L Na₂-EDTA*2H₂O, 1 g/L CaSO₄*2H₂O, 0.3 g/L ZnSO₄*7H₂O, 7.4 g/L (NH₄)₂Fe(SO₄)₂, 0.2 g/L MnSO₄*H₂O, 0.15 g/L CuSO₄*5H₂O, 0.04 g/L Na2MoO₄*2H₂O, 0.04 g/L Na₂SeO₄, 10 mg/L thiamin*HCl, 0.1 mg/L vitamin B12, 1 mM IPTG, pH 7.0) in a 100 mL baffled shake flask were inoculated with an overnight culture (grown on the above described medium without lactose and IPTG) of a 2′-FL producing strain as in example 1 to a start OD of 0.5 and incubated for 24 hours in the above described medium including lactose and IPTG as given above at 200 rpm at 37° C. Samples were taken and analyzed for carbon utilization and product formation. Similarly, the 2′-FL producing strain with crr deletion was cultured sampled and analyzed.

TABLE 6
Carbon-conversion-efficiency and carbon substrate
flexibility for 2′-FL producing strains with
wt crr or crr functional gene deletion (delta crr)

| | Carbon- | Carbon- | Carbon- | Carbon- |
|---|---|---|---|---|
| | conversion- | conversion- | conversion- | conversion- |
| | efficiency | efficiency | efficiency | efficiency |
| | (g Fucose/ | (g Fucose/ | (g Fucose/ | (g Fucose/ |
| | g fructose) | g mannose) | g glucose) | g glycerol) |
| | relative to | relative to | relative to | relative to |
| | wildtype | wildtype | wildtype | wildtype |
| | (=100%) | (=100%) | (=100%) | (=100%) |
| Crr wt | 100 | 100 | 100 | 100 |
| delta | 175 | 116 | 133 | 166 |
| crr | | | | |

1-15. (canceled)

16. Method to increase the carbon substrate flexibility of the production of and/or to increase the carbon-conversion-efficiency of and/or to increase the space-time-yield of one or more fine chemicals produced by a host organism suitable for the production of one or more fine chemicals including the steps of increasing the Adenosine 3′,5′-cyclic monophosphate (cAMP, CAS Number: 60-92-4) levels of the host organism compared to the non-modified host organisms, maintaining the host organism in a setting allowing it to grow, growing the host organisms in the presence of substrates and under conditions suitable for the production of one or more fine chemicals and optionally separating one or more fine chemicals from the host organism or remainder thereof.

17. Method according to claim 16, wherein the cAMP level of the host organism is increased by
a. Inactivating the regulatory activity found in a wildtype adenylate cyclase, and/or
b. generating a mutated adenylate cyclase lacking the regulatory activity found in a wildtype adenylate cyclase, and/or
c. introduction into the host organism of a mutated adenylate cyclase lacking the regulatory activity found in a wildtype adenylate cyclase.

18. Method according to claim 16 wherein the cAMP level of the host organism is increased in an inducible manner and the increase is compared to the host organisms without induction.

19. Method according to claim 17, wherein the mutated adenylate cyclase is introduced by introduction of a transgene.

20. Method according to claim 17, wherein the mutated adenylate cyclase or the adenylate cyclase with inactivated regulatory activity has a deletion compared to wildtype form of the adenylate cyclase of the host organisms.

21. Method according claim 20, wherein the deletion is removing the regulatory part of the adenylate cyclase without disrupting the part producing cAMP.

22. Method according to claim 20, wherein the deletion is a deletion of the regulatory part of the protein that corresponds to C-terminal part of the adenylate cyclase encoded by an Escherichia coli cyaA gene, preferably that part that corresponds to the C-terminal part of the CyaA protein as provided in SEQ ID NOS:19 or 20, or an adenylate cyclase protein of at least 80% sequence identity to positions 1 to 412.

23. The method according to claim 16, wherein the method includes the step of supplying the host organism with a carbon source, wherein the carbon source is a complex or a defined carbon source or combinations thereof.

24. The method according to claim 16, wherein the host organism is a genetically modified microorganism cell and wherein preferably the one or more fine chemical is one or more oligosaccharide and wherein the method includes before the growth of the genetically modified microorganism the step of inactivating or removing in the genetically modified microorganism the Crr protein or the endogenous protein(s) corresponding to the Crr protein in E. coli (SEQ ID NO: 26).

25. Modified host cell suitable for the production of a fine chemical wherein the host cell is able to grow on glycerol and/or glucose and/or maltose and/or fructose and/or sucrose, preferably sucrose, glycerol, glucose and/or fructose, wherein the modified host cell comprises an adenylate cyclase with inactivated or absent regulatory activity, that has adenylate cyclase activity, and wherein the host organism has increased cAMP level compared to a non-modified host cell, wherein the non-modified host cell is unable to grow substantially on glycerol and/or glucose and/or maltose and/or fructose and/or sucrose.

26. Modified host cell of claim 25, wherein at least one adenylate cyclase protein corresponding to the protein encoded by the cyaA gene of Escherichia coli is lacking a regulatory activity, preferably lacking the part that corresponds to C-terminal part of the CyaA protein as provided in SEQ ID NOS:19 or 20, or an adenylate cyclase protein of at least 80% sequence identity to positions 1 to 412.

27. Modified host cell of any of claim 25, wherein the host cell is a genetically modified microorganism for an enhanced production of oligosaccharides, wherein said genetically modified microorganism is capable to produce oligosaccharides, wherein said genetically modified microorganism comprises functional genes coding for a PTS carbohydrate utilization system, wherein in said genetically modified microorganism the abundance and/or activity of the Crr protein (SEQ ID NO: 26), of variants thereof or of endogenous protein corresponding to the Crr protein in said microorganism is decreased, and wherein the space-time-yield, carbon substrate flexibility or carbon-conversion-efficiency of oligosaccharide production by the genetically modified microorganism is increased compared to a control with unaltered abundance and/or activity of the Crr protein (SEQ ID NO: 26), of variants thereof or of endogenous protein(s) corresponding to the Crr protein.

28. Modified host cell of claim 25, wherein the host cell is a genetically modified microorganisms and the gene encoding the Crr protein, variants thereof or the endogenous protein(s) corresponding to the Crr protein in said microorganism is attenuated or deleted in said genetically modified microorganism.

29. Claim 16 wherein at least one fine chemical is a human milk oligosaccharide.

30. Claim 16 wherein space-time-yield, carbon substrate flexibility and/or carbonconversion-efficiency of the production of one or more fine chemicals, preferably one or more oligosaccharides, is increased by at least 20% compared to the controls.

15. Use of
a. an adenylate cyclase protein with inactive regulatory domain and functional catalytic domain to produce cAMP as defined in claim 16; and/or
b. inactivation and/or the reduction in abundance of the Crr protein or the endogenous protein corresponding to the Crr protein in E. coli (SEQ ID NO: 26)
to increase in a host cell the carbon substrate flexibility of the production of and/or to increase the carbon-conversion-efficiency of and/or to increase the space-time-yield of one or more human milk oligosaccharides.
with unaltered abundance and/or activity of the Crr protein (SEQ ID NO: 26), of variants thereof or of endogenous protein(s) corresponding to the Crr protein.

13. Modified host cell of any of claims 10 to 12, wherein the host cell is a genetically modified microorganisms and the gene encoding the Crr protein, variants thereof or the endogenous protein(s) corresponding to the Crr protein in said microorganism is attenuated or deleted in said genetically modified microorganism.

14. Any of the preceding claims wherein at least one fine chemical is a human milk oligosaccharide.

15. Any of the preceding claims wherein space-time-yield, carbon substrate flexibility and/or carbon-conversion-efficiency of the production of one or more fine chemicals, preferably one or more oligosaccharides, is increased by at least 20% compared to the controls.
